(19) United States
(12) Patent Application Publication
(10) Pub. No.: US 2025/0179162 A1
(43) Pub. Date: Jun. 5, 2025

(54) ANTIGEN BINDING FRAGMENTS CONJUGATED TO A PLURALITY OF FC ISOTYPES AND SUBCLASSES

(71) Applicant: BIO-RAD ABD SEROTEC GMBH, NEURIED (DE)

(72) Inventor: HANS JOACHIM KNAPPIK, MOORENWEIS (DE)

(21) Appl. No.: 19/061,075

(22) Filed: Feb. 24, 2025

Related U.S. Application Data
(63) Continuation of application No. 16/822,083, filed on Mar. 18, 2020.
(60) Provisional application No. 62/819,748, filed on Mar. 18, 2019.

(51) Int. Cl. C07K 16/18

(52) U.S. Cl. CPC C07K 16/18; C07K 2317/52; C07K 2317/55; C07K 2319/21; C07K 2319/43; C07K 2319/70

(57) ABSTRACT

The invention pertains to a plurality of full-length antibodies, wherein each full-length antibody comprises an antigen binding fragment specifically binding to a unique antigen and comprises a first binding motif at the C-terminus and an Fc fragment belonging to a unique combination of species, isotype and subclass and comprises a second binding motif at the N-terminus, wherein the first binding motif and the second binding motif for each antibody are covalently conjugated to each other via protein ligation. Assays for detecting a plurality of antigens in a sample by contacting the sample with the plurality of full-length antibodies are also provided. Further provided are nucleic acid constructs encoding the plurality of full-length antibodies.

### CROSS-REFERENCE TO RELATED APPLICATIONS

This application is a continuation of U.S. application Ser. No. 16/822,083, filed Mar. 18, 2020, which claims the benefit of U.S. Provisional Application 62/819,748 filed on Mar. 18, 2019 which is hereby incorporated by reference in its entirety.

### REFERENCE TO SEQUENCE LISTING

The Sequence Listing for this application is labeled “Seq-List.xml” which was created on Feb. 11, 2025 and is 63,737 bytes. The entire content of the sequence listing is incorporated herein by reference in its entirety.

### BACKGROUND OF THE INVENTION

Antibodies isolated from libraries, such as PCR-derived, semi-synthetic or fully synthetic libraries, by in vitro selection technologies including phage display, bacterial display or ribosome display are typically expressed as antigen binding fragments (e.g., scFv or Fab). This is because the bacterial expression systems typically do not allow expression of functional full-length antibodies. Additional reasons to prepare antibody libraries containing antigen binding fragments include the selection of desirable antibodies based on intrinsic binding affinity and not on avidity caused by the bivalent nature of antibodies. After a typical selection experiment to identify desirable antigen binding fragments (e.g., panning), the enriched pool of genes encoding the desired antibodies is sub-cloned into a bacterial expression vector for further analysis.

Antigen binding fragments isolated from such libraries often need to be converted later into full-length antibodies of different isotypes and subclasses, such as human IgG1-4, IgA, IgE, or IgM or into antibodies with Fc regions from different species, such as murine, rat, rabbit, goat or chicken. Full-length antibodies containing desirable antigen binding fragments are produced for specific practical applications of the antigen binding fragments.

For example, full-length antibodies can be used as positive controls or calibrators in a diagnostic assay in which a patient sample is measured for the presence of antibodies against a given target. This is often performed during the diagnosis of infectious diseases or autoimmune diseases. In these situations, the positive control antibodies need to contain the Fc fragments of the antibodies that will be detected, for instance IgG1 or IgE or IgM, to enable the isotype and subclass-specific anti-Fc detection reagents binding to the control antibody. Alternatively, full-length antibodies with an Fc from a pre-defined species and with a pre-defined isotype can be used in combination with other antibodies from different species or with different isotypes in the same experiment, i.e., multiplexing, for instance in Western blotting or IHC experiments, if species-specific or isotype-specific secondary antibodies are used for detection.

Conversion of antigen binding fragments to full-length antibodies and subsequent production consists of several steps and is a laborious process, which typically takes several weeks. First, the genes encoding antibody variable domains are typically re-synthesized to adopt the codon usage to the mammalian expression system used for antibody production. Sometimes, potential glycosylation sites are present in the CDR regions of the selected antigen binding fragment and need to be removed by site-directed mutagenesis before expression in eukaryotic cells. Such mutation in the CDR region may change antibody affinity or specificity. Second, the synthesized variable heavy (VH) and variable light (VL) gene fragments are cloned into a mammalian expression vector that contains the necessary gene fragments encoding the antibody constant regions (CL and CHI-hinge-CH2-CH3 for IgG1, for example). Third, the plasmid DNA is prepared and used to transfect a suitable mammalian cell line. Fourth, the transfected cell line is expanded in culture for several days, until the antibody containing supernatant can be harvested. Fifth, the antibody is purified from the cell culture supernatant. If the same antigen binding site is needed as full-length antibody with several isotypes and subclasses (e.g., IgG1 and IgG2) or with Fc fragments from several species, the cloning and expression steps have to be repeated for each type.

### Protein Ligation

Several technologies enable covalent conjugation of polypeptides at specific pre-determined sites. One example is the sortase system (Schmohl et al., 2014), whereby a short peptide (the sorting motif) is genetically fused to the C-terminus of one polypeptide and two glycine residues are genetically fused to the N-terminus of a second polypeptide (or vice versa). In the presence of the sortase enzyme, the two modified polypeptides are fused together. Other enzymatic protein ligase systems are based on butelase (Nguyen et al., 2014) or peptiligase (Toplak et al., 2016).

Another example is the in-frame addition of nucleotides encoding one or more cysteines to the C-or N-termini of two polypeptides. When such free cysteine containing polypeptides are mixed under oxidizing conditions, they will form disulfide bridges. Such systems, however, suffer from the synthesis of many side-products and from instability of the disulfide bridge under reducing conditions.

A third example is the so-called SpyTag/SpyCatcher (Reddington et al., 2015) system. Here, the concept of spontaneous isopeptide formation in naturally occurring proteins has been used to covalently attach one polypeptide to another. A domain from the Streptococcus pyogenes protein FbaB, which contains such isopeptide bond is split into two parts. One part, the SpyTag, is a 13 amino acid peptide that contains part of the autocatalytic center. The other part, the SpyCatcher, is a 116 amino acid protein domain containing the other part of the center. Mixing those two polypeptides restores the autocatalytic center and leads to formation of the isopeptide bond, thereby covalently linking the SpyTag (SEQ ID NO: 7) to the SpyCatcher (SEQ ID NO: 8), see Zakeri et al., 2012. Further engineering has led to a shorter version of SpyCatcher with only 84 amino acids (SEQ ID NO: 9) as well as optimized versions, SpyTag002 (SEQ ID NO: 34) and SpyCatcher002 (SEQ ID NO: 28) (Li et al., 2014 and Keeble et al., 2017) and SpyTag003 (SEQ ID NO: 43) and SpyCatcher003 (SEQ ID NO: 44) with accelerated reaction (Keeble et al., 2019); both of which are hereby incorporated by reference in their entirety. A further modification of the system was the invention of SpyLigase (Fierer et al., 2014), which was achieved by splitting the FbaB domain into three parts, the SpyTag, the K-tag and the SpyLigase. SpyTag and K-tag are both short peptides that are covalently fused by addition of SpyLigase.

Applications of such system includes stabilization of proteins by circularization, vaccine generation, multimerization of proteins by integrating streptavidin/biotin with SpyTag/SpyCatcher (Reddington et al., 2015), affibody and Fab multimerization (Fierer et al., 2014), generation of antibodies from modules (Alam et al., 2017) as well as creation of antibody-drug conjugates (Siegmund et al., 2016), and generation of bispecific antibodies (Yumura et al., 2017). A similar system using the adhesin RrgA from Streptococcus pneumoniae was developed and termed SnoopTag/SnoopCatcher (Veggiani et al., 2016), with a later development of a SnoopLigase system (Buldun et al., 2018). The SnoopTag (SEQ ID NO: 35)/SnoopCatcher (SEQ ID NO: 36) technologies are hereby incorporated by reference in their entirety.

### BRIEF SUMMARY OF THE INVENTION

Certain embodiments of the invention provide a full-length antibody comprising an antigen binding fragment comprising a first binding motif at the C-terminus and an Fc fragment comprising a second binding motif at the N-terminus (the term “FcCatcher” is used in the Examples section to describe this construct), wherein the first binding motif and the second binding motif are capable of covalent conjugation to each other via protein ligation and wherein the antigen binding fragment and the Fc fragment are obtained from different species. Specific embodiments of the invention also provide a plurality of full-length antibodies, wherein each full-length antibody comprises an antigen binding fragment specifically binding to a unique antigen and comprises a first binding motif at the C-terminus and an Fc fragment belonging to a unique combination of species, isotype and subclass and comprises a second binding motif at the N-terminus, wherein the first binding motif and the second binding motif for each antibody can be covalently conjugated to each other via protein ligation.

Specific embodiments of the invention provide a plurality of Fc fragments, each Fc fragment comprising a binding motif at the N-terminus, wherein the binding motif can be covalently conjugated to a suitable antigen binding fragment, which bears another binding motif at its C-terminus, via protein ligation. Also, within the plurality of Fc fragments, each Fc fragment belongs to a unique combination of species, isotype and/or subclass. By using such a plurality of Fc fragments, full length antibodies or full length antibody-like structures can be generated via protein ligation of the Fc fragments and antigen binding fragments, each of which contains an appropriate binding motif. The resulting populations of full-length antibodies can be utilized in a variety of applications, such as multiplex immunoassays.

The first and the second binding motifs that facilitate the formation of a covalent linkage between the Fc fragment and the antigen binding fragment include SpyTag sequences, SpyCatcher sequences, SnoopTag sequences, SnoopCatcher sequence, Isopeptag/Split Spy0128, SdyTag/SdyCatcherDANG short, SpyLigase, SnoopLigase, Sortase motifs, butelase substrates, and peptiligase substrates. Assays for detecting a plurality of antigens in a sample by contacting the sample with the plurality of full-length antibodies are also provided. Further provided are nucleic acid constructs encoding the plurality of full-length antibodies.

### BRIEF DESCRIPTION OF THE DRAWINGS

The patent or application file contains at least one drawing executed in color. Copies of this patent or patent application publication, with color drawing(s), will be provided by the Office upon request and payment of the necessary fee.

FIG. 1 shows an image of a Coomassie-stained SDS-PAGE gel of the kinetics of product formation when ligating Fab-SpyTag2 with human IgG1-FcCatcher3 as described in Example 3 .

FIG. 2 shows the results of an ELISA titration experiment as described in Example 4 with a Fab-SpyTag2 antibody ligated to hlgG1-FcCatcher3 compared to the same antibody in human IgG1 format. A human anti-Fc: HRP secondary antibody was used for detection.

FIG. 3 shows an image of a Coomassie-stained SDS-PAGE gel of the reduced products of the ligation reaction of Fab-SpyTag with FcCatchers from various species as described in Example 5.

FIG. 4 shows a scheme of the three primary and secondary antibodies used in the immune-fluorescence study, and the resulting immunofluorescence images of staining of U2OS cells, as described in Example 5.

FIG. 5 shows flow cytometry data of Jurkat cells co-stained with a mouse anti-CD3 Fab-SpyTag2/hIgG1-FcCatcher3 ligation product and mouse anti-CD45 Fab-SpyTag2/rbIgG-FcCatcher3 ligation product together with fluorescence-labelled anti-human and anti-rabbit anti-Fc secondary antibodies as described in Example 6.

### DETAILED DISCLOSURE OF THE INVENTION

The invention implements protein ligation to circumvent the steps currently needed for production of full-length antibodies from antigen binding fragments. The invention provides modified Fc fragments that are equipped with a motif that allows site-specific protein conjugation. Such modified Fc fragments can be prepared from Fc sequence information from various species and isotypes and subclasses, e.g., human IgG1, mouse IgG2a, or rabbit IgG, etc.

Before conjugation with antigen binding fragments, the modified Fc fragments can also be conjugated to suitable labels, such as fluorescent dyes or detection enzymes like HRP.

Accordingly, certain embodiments of the invention provide a full-length antibody comprising an antigen binding fragment comprising a first binding motif at the C-terminus and an Fc fragment comprising a second binding motif at the N-terminus, wherein the first binding motif and the second binding motif are covalently conjugated to each other via protein ligation. To produce such full-length antibody, an antigen binding fragment is produced with a first binding motif at its C-terminus and an Fc fragment is produced with a second binding motif at its N-terminus and the antigen binding fragment-first binding motif fusion protein is mixed with the Fc fragment-second binding motif fusion protein under appropriate conditions to facilitate protein ligation of the first binding motif and the second binding motif to produce the full-length antibody. The full-length antibody so produced can be further purified. For example, the reaction may produce antibodies containing only one Fab attached to Fc. Such by-products can be removed by, for instance, size-exclusion chromatography or affinity chromatography using Protein A column or a column specifically binding to a tag introduced onto the Fc or Fab fragment.

Typically, the antigen binding fragment is obtained from a first species and the Fc fragment is obtained from a second species that is different from the first species. For example, if the antigen binding fragment is obtained from a human antibody, the Fc fragment can be obtained from a murine antibody. Any combination of different species can be used. In preferred embodiments, the antigen binding fragments attached to an Fc fragment bind to the same epitope. The antigen binding fragments and Fc fragments can be derived from humans, non-human primates, rodents such as mice and rats, rabbit, hamster, goat, sheep, bovine, porcine, equine, canine, feline, and camelid. Additional species that could be used in the instant invention are well known in the art and such embodiments are within the purview of the invention.

In certain embodiments, the invention provides a plurality of full-length antibodies that can be used in the same assay reaction, i.e., for multiplexing. Accordingly, such embodiments provide a plurality of full-length antibodies, wherein each full-length antibody comprises an antigen binding fragment comprising a first binding motif at the C-terminus and an Fc fragment comprising a second binding motif at the N-terminus, wherein the first binding motif and the second binding motif are covalently conjugated to each other via protein ligation. Also, within a set of the plurality of full-length antibodies, each antigen binding fragment specifically binds to a unique antigen and each Fc fragment belongs to a unique species, isotype and subclass. In such an embodiment, the antigen binding fragments and the Fc fragments may be heterologous (derived from different human or non-human animal species, e.g., human antigen binding fragments covalently conjugated to a murine Fc fragment via binding motifs) or homologous (derived from the same species, e.g., human antigen binding fragments covalently conjugated to human Fc fragments via the binding motifs).

In further embodiments, the invention provides an antigen binding fragment and an Fc fragment, wherein the antigen binding fragment and the Fc fragment can be conjugated to form a full-length antibody. In certain embodiments, the antigen binding fragment comprises a first binding motif at the C-terminus and the Fc fragment comprises a second binding motif at the N-terminus, wherein the first binding motif and the second binding motif can be covalently conjugated to each other via protein ligation when contacted with each other under appropriate conditions. The antigen binding fragment and/or the Fc fragment can be conjugated to a detectable label, particularly, an optical label.

To produce a full-length antibody, an antigen binding fragment containing a first binding motif at its C-terminus is mixed with the Fc fragment comprising a second binding motif at the N-terminal. Such mixing is performed under appropriate conditions that facilitate protein ligation of the first binding motif and the second binding motif. The full-length antibody so produced can be further purified. For example, the reaction may produce antibodies containing only one Fab attached to Fc. Such by-products can be removed by, for instance, size-exclusion chromatography or affinity chromatography using Protein A column or a column specifically binding to a tag introduced onto the Fc or Fab fragment.

Also provided are kits for producing a full-length antibody. Such a kit can contain an antigen binding fragment comprising a first binding motif at the C-terminus and the Fc fragment comprising a second binding motif at the N-terminus. The antigen binding fragment and/or the Fc fragment can be conjugated to a detectable label, particularly, an optical label. Accordingly, in some embodiments, the kit comprises two or more of the following components:

1. an antigen binding fragment containing a first binding motif at its C-terminus, optionally comprising a detectable label (e.g., biotin, HRP, or a fluorophore); and
2. an Fc fragment comprising a second binding motif at the N-terminus, optionally comprising a detectable label (e.g., biotin, HRP, or a fluorophore); and/or
3. a nucleic acid construct comprising a polynucleotide sequence encoding an antigen binding fragment and/or an Fc fragment as defined in 1 and/or 2,
wherein the first binding motif and the second binding motif are capable of covalent conjugation to each other via protein ligation.

A kit user can mix the antigen binding fragment and the Fc fragment under appropriate conditions where the antigen binding fragment and the Fc fragment will interact with each other via protein ligation, either spontaneously or with the help of an enzyme, to form a covalent bond. A kit user can also use a nucleic acid construct comprising a polynucleotide sequence encoding the antigen binding fragment and/or the Fc fragment to express any of the peptides in a suitable host.

Each of the components of the kit can be provided in liquid form (e.g., as a solution) or as a solid (e.g., a powder) that is reconstituted with liquid, e.g., buffer, prior to use. In some embodiments, the kit further comprises instructions for ligating one or more of the binding pairs.

The term “label” or “detectable label” refers to a composition detectable by spectroscopic, photochemical, biochemical, immunochemical, chemical, or other physical means. For example, useful labels include fluorescent dyes (fluorophores), fluorescent quenchers, luminescent agents, electron-dense reagents, enzymes (e.g., as commonly used in an ELISA), biotin, digoxigenin, 32P and other isotopes, haptens, proteins, nucleic acids, or other substances which can be made detectable, e.g, by incorporating a label into an oligonucleotide or peptide. The term includes combinations of single labeling agents, e.g., a combination of fluorophores that provides a unique detectable signature, e.g., at a particular wavelength or combination of wavelengths.

Exemplary detectable labels include, but are not limited to, a fluorophore, a fluorescent protein such as green fluorescent protein (GFP), biotin, an enzyme such as horse radish peroxidase (HRP) or other peroxidases, alkaline phosphatase, luciferase, and a split fluorescent protein (e.g., split GFP) or enzymes (e.g., NanoLuc® Binary Technology from Promega). Exemplary fluorophores include, but are not limited to, Alexa dyes (e.g., Alexa 350, Alexa 430, Alexa 488, etc.), AMCA, BODIPY 630/650, BODIPY 650/665, BODIPY-FL, BODIPY-R6G, BODIPY-TMR, BODIPY-TRX, Cascade Blue, Cy2, Cy3, Cy5, Cy5.5, Cy7, Cy7.5, Dylight dyes (Dylight405, Dylight488, Dylight549, Dylight550, Dylight 649, Dylight680, Dylight750, Dylight800), 6-FAM, fluorescein, FITC, HEX, 6-JOE, Oregon Green 488, Oregon Green 500, Oregon Green 514, Pacific Blue, REG, Rhodamine Green, Rhodamine Red, ROX, R-Phycoerythrin (R-PE), Starbright Blue Dyes (e.g., Starbright Blue 520, Starbright Blue 700), TAMRA, TET, Tetramethylrhodamine, Texas Red, and TRITC.

The phrase “antigen binding fragment” as used herein refers to the antigen binding portion of an antibody, such as Fab. Other antigen binding fragments include variable fragment (Fv), single chain variable fragment (scFv) or variable domain of heavy chain antibodies (VHH). Further examples of antigen binding fragments include monovalent forms of antigen binding fragments that contain the antigen binding site include single chain Fab fragment (scFab), single domain antibody (sdAbs), Shark Variable New Antigen Receptor (VNAR) or Variable Lymphocyte Receptors (VLRs). Furthermore, binding reagents derived from non-antibody scaffolds such as affimers, affibodies, darpins, anticalins, monobodies are also considered “antigen binding fragments”. Additional examples of antigen binding fragments are well known in the art and uses of such antigen binding fragments are within the purview of the instant invention.

The phrase “each Fc fragment belongs to a unique combination of species, isotype and subclass” indicates that an Fc fragment of a full-length antibody within the set of the plurality of full-length antibodies belongs to a specific combination of species, isotype, subclass and allotype and no other Fc fragment from the plurality of full-length antibodies has the same combination of species, isotype and subclass. Thus, if the set of the plurality of full-length antibodies contains fifty full-length antibodies, each of the fifty antibodies has an Fc fragment belonging to a different combination of species, isotype, subclass or allotype compared to the remaining forty-nine Fc fragments.

Typically, a given species produce several types of antibodies. For example, a human or a mouse is capable of producing five antibody heavy-chain related isotypes, namely, IgA, IgD, IgE, IgG and IgM. Each isotype may further contain several subclasses. For example, human IgG has four subclasses, namely, IgG1, IgG2, IgG3 and IgG4. Thus, a species can contain a number of antibody isotypes and several subclasses within each isotype. A skilled artisan can identify and select appropriate isotypes subclasses from appropriate species for use in the instant invention. For example, a set of five full-length antibodies can comprise five Fc fragments, for example, human IgG1, human IgG2, human IgA, mouse IgG3, and mouse IgE. Furthermore, a certain subclass (e.g., IgG1) may contain a number of allotypes, which are variants of this subclass in the genetic pool of a species. For instance, the human IgG1 subclass contain the allotypes G1m(za), Glm(f), G1m(fa), G1m(zax) and G1m zav) that can be distinguished at the amino acid sequence. Within IgG1, most amino acid differences are located in the CH3 domain. Fc fragments with different allotypes may be used in the invention, for instance if the reagents used to detect the antibodies are allotype-specific.

As noted above, the set of full length antibodies can be used in a multiplex assay, i.e., the plurality of full-length antibodies can be used to detect a plurality of antigens in an assay. Therefore, within the plurality of full-length antibodies, each antigen binding fragment specifically binds to a unique antigen and each Fc-region can be detected with a species-or isotype-or subclass-or allotype-specific secondary reagent.

The phrase “each antigen binding fragment specifically binds to a unique antigen” indicates that an antigen binding fragment of a full-length antibody within the set of the plurality of full-length antibodies specifically binds a specified antigen and no other full-length antibody from the plurality of full-length antibodies binds to the same antigen. Thus, if a set of a plurality of full-length antibodies contains fifty full-length antibodies, each of the fifty antibodies specifically binds to a different antigen as compared to the remaining forty-nine full-length antibodies.

Thus, in certain embodiments, the invention provides a plurality of full-length antibodies, wherein each of the full-length antibodies in the set is conjugated to a unique label. In this embodiment, the Fc fragment can be identical for the plurality of full-length antibodies and antigen (or epitope) specific antibodies are distinguished by way of a unique label. Thus, a full-length antibody within the plurality of full-length antibodies is conjugated to a label and no other the full-length antibody from the plurality of full-length antibodies has the same label or specificity for the same antigen (or epitope). Thus, if the set of the plurality of full-length antibodies contains fifty full-length antibodies, each of the fifty antibodies has a different label and specificity for a different antigen (or epitope). Presence of unique label on each of the full-length antibodies facilitates the quantification of unique Fc fragments and consequently, the quantification of the unique antigen to which the full-length antibody is bound. In a one particular embodiment, the label can be a unique bead. For example, a combination of unique beads is provided by the Bio-Plex® multiplex immunoassay system, which provides multiplexing of up to 100 different assays within a single sample. The use of different colored beads enables the simultaneous multiplex detection of many full-length antibodies and consequently, many antigens in the same sample.

In even further embodiments, the invention provides a plurality of full-length antibodies, each of the plurality of antibodies having an Fc fragment that permits one to distinguish between the antibodies on the basis of the Fc fragment by using secondary antibodies specific for the unique Fc fragments. Therefore, in a multiplexing reaction, different secondary antibodies can be used to distinguished different Fc fragments, and hence, the different antigens that the different antibodies recognize.

The unique Fc fragments in the plurality of full-length antibodies can be detected by secondary antibodies to the unique Fc fragments. Thus, certain embodiments of the invention further comprise a plurality of secondary antibodies against the plurality of unique Fc fragments, wherein each secondary antibody specifically binds to a unique Fc fragment from a particular species, subtype and subclass (and possibly allotype). Each secondary antibody can be conjugated to a unique detectable label, wherein the unique label on each of the secondary antibodies facilitates the quantification of unique Fc fragments and consequently, the quantification of unique antigen to which the full-length antibody is bound.

Alternatively, certain embodiments of the invention further comprise a plurality of secondary antibodies against the plurality of unique Fc fragments, wherein each secondary antibody specifically binds to a unique Fc fragment from a particular species, subtype and subclass, and wherein each secondary antibody is conjugated to a unique bead.

An example of a combination of unique beads is provided by the Bio-Plex® multiplex immunoassay system. The technology enables multiplex immunoassays in which one secondary antibody against a unique Fc fragment is attached to a set of beads with the same color and such secondary antibody attached to the unique set of beads can be visualized, for example, by using a detectable label. The use of different colored beads enables the simultaneous multiplex detection of many antigens in the same sample.

In certain embodiments, the invention provides a plurality of Fc fragments comprising a binding motif at their N-termini. Such plurality of Fc fragments can be used to produce a customizable set of full-length antibodies that bind to a single antigen, or multiple antigens, of interest. For example, a plurality of antigen binding fragments of interest can then be expressed with suitable binding motifs at their C-termini and such antigen binding fragments could be mixed with appropriate Fc fragments to produce a customized plurality of full-length antibodies. Thus, certain embodiments of the invention provide a plurality of Fc fragments, each Fc fragment comprising a binding motif at the N-terminus, wherein the binding motif can be covalently conjugated to an antigen binding fragment having a suitable binding motif via protein ligation. Also, within the plurality of Fc fragments, each Fc fragment can belong to the same species, isotype and subclass or be unique (e.g., from a unique species, isotype or subclass).

A specific embodiment of the invention provides a plurality of antigen binding fragments, wherein each antigen binding fragment is fused at the C-terminus to a first binding motif, and each antigen binding fragment specifically binds to the same antigen but recognizes a different epitope on the antigen and/or has a different antigen binding affinity. Each of the first binding motifs from the plurality of antigen binding motifs can form a covalent bond with a second binding motif present at the N terminus of Fc fragments when brought into contact with one another either spontaneously or with the help of an enzyme. This embodiment provides a “synthetic polyclonal antibody preparation”, i.e., a preparation of antibodies that specifically bind to the same antigen; however, each antibody in the preparation recognizes different epitope and/or has a different binding affinity. As discussed above, each Fc fragment can belong to the same species, isotype and subclass or be unique (e.g., from a unique species, isotype or subclass).

Further embodiments of the invention provide a method of producing a plurality of full-length antibodies. Such method comprises contacting one antigen binding fragment from a plurality of antigen binding fragments with one Fc fragment from a plurality of Fc fragments, wherein the each of the plurality of antibody finding fragments comprises at its C-terminus a first binding motif and each of the plurality of Fc fragments comprises at its N-terminus a second binding motif. The conditions for contacting the antigen binding fragments and the Fc fragments are such that a covalent bond is formed between the first binding motif and the second binding motif. Thus, a plurality of full-length antibodies are produced, each antibody comprising an antigen binding fragment comprising a first binding motif at the C-terminus and an Fc fragment comprising a second binding motif at the N-terminus.

As discussed above, several technologies enable covalent conjugation of polypeptides at specific pre-determined sites via “protein ligation”. The term “protein ligation” as used herein refers to conjugation via a covalent bond of a first binding motif on a first protein to a second binding motif on a second protein, wherein the conjugation occurs when the first binding motif and the second binding motif are brought into contact with one another and the covalent bond is formed either spontaneously under suitable conditions or with the help of an enzyme. Typically, the first binding motif is present at the C-terminus of a first protein and the second binding motif is present at the N-terminus of the second protein. Usually, the first protein is expressed as a fusion protein with the first binding motif at its C-terminus and the second protein is expressed as a fusion protein with the second binding motif at its N-terminus.

The term “first binding motif” refers to a peptide sequence that is attached to the C-terminus of an antigen binding fragment and that facilitates the formation of a covalent linkage via protein ligation to a second binding motif present at the N-terminus of an Fc fragment. Similarly, the term “second binding motif” refers to a peptide sequence that is attached to the N-terminus of an Fc fragment and that facilitates the formation of a covalent linkage via protein ligation to a first binding motif present at the C-terminus of an antigen binding fragment.

As noted above, “protein ligation” refers to a covalent bond formation, either spontaneously or with the help of an enzyme, between the first binding motif and the second binding motif when these motifs are brought into contact with one another. Also, as discussed throughout this disclosure, protein ligation occurs between specific combinations of peptide sequences, for example, between SpyTag and SpyCatcher, SnoopTag and SnoopCatcher, sortase recognition domain and sortase bridging domain, butelase recognition motif and the amino terminus of another polypeptide, SpyTag002 (SEQ ID NO: 34) and SpyCatcher002 (SEQ ID NO: 28), SpyTag (SEQ ID NO: 29) and K-Tag (SEQ ID NO: 33), SpyTag003 (SEQ ID NO: 43) and SpyCatcher003 (SEQ ID NO: 44), etc.

Therefore, to produce a full-length antibody of the instant invention, the first binding motif present at the C-terminus of an antigen binding fragment is capable of forming a covalent bond via protein ligation to the second binding motif at the N-terminus of an Fc fragment. For example, if a first binding motif present at the C-terminus of an antigen binding fragment is a SpyTag, or the improved versions SpyTag002 or SpyTag003, the corresponding second binding motif present at the N-terminus of an Fc fragment is a SpyCatcher, or the improved versions SpyCatcher002 or SpyCatcher003. Alternatively, if a first binding motif present at the C-terminus of an antigen binding fragment is a SpyCatcher, or the improved versions SpyCatcher002 or SpyCatcher003, the corresponding second binding motif present at the N-terminus of an Fc fragment is a SpyTag, or the improved versions SpyTag002 (SEQ ID NO: 34) or SpyTag003 (SEQ ID NO: 43).

Similarly, if a first binding motif present at the C-terminus of an antigen binding fragment is a SnoopTag (SEQ ID NO: 35) or a sequence at least 70% identical to SEQ ID NO: 35, the corresponding second binding motif present at the N-terminus of an Fc fragment is a SnoopCatcher (SEQ ID NO: 36) or a sequence at least 50% identical to SEQ ID NO: 36. Alternatively, if a first binding motif present at the C-terminus of an antigen binding fragment is a SnoopCatcher (SEQ ID NO: 36) or a sequence at least 50% identical to SEQ ID NO: 36, the corresponding second binding motif present at the N-terminus of an Fc fragment is a SnoopTag (SEQ ID NO: 35) or a sequence at least 70% identical to SEQ ID NO: 35.

Further, in the case of enzymatic protein ligation, if a first binding motif present at the C-terminus of an antigen binding fragment is a SpyTag, the corresponding second binding motif present at the N-terminus of an Fc fragment is a K-Tag. Alternatively, if a first binding motif present at the C-terminus of an antigen binding fragment is a K-Tag, the corresponding second binding motif present at the N-terminus of an Fc fragment is a SpyTag. In both cases a SpyLigase is needed to catalyze the isopeptide bond formation between the two tags.

As such, a first binding motif present at the C-terminus of an antigen binding motif and a second binding motif present at the N-terminus of an Fc fragment are selected such that the two motifs interact with each other via protein ligation, either spontaneously or with the help of an enzyme, to form a covalent bond.

The expression of these proteins can be carried out in suitable host cells, including prokaryotic cells, such as Escherichia coli or eukaryotic cells, such yeast or CHO cells. Suitable techniques for expression of fusion proteins are known to a person of ordinary skill in the art and such embodiments are within the purview of the invention.

Thus, a protein ligation system comprises attaching a first binding motif to a first protein and attaching a second binding motif to a second protein and covalently joining the first protein and the second protein via covalent binding between the first binding motif and the second binding motif. Such covalent binding can be autocatalytic, i.e., catalyzed by the interaction between the first binding motif and the second binding motif. The covalent binding can also be enabled by enzymes that catalyze such binding reactions.

Non-limiting examples of the protein ligation include the sortase system, butelase system, peptiligase system, cysteine mediated disulfide bridge formation, SpyTag/SpyCatcher system, SpyTag with the shorter version of SpyCatcher, SpyTag002/SpyCatcher002 or SpyTag003/SpyCatcher003 systems with accelerated reaction; SpyTag/K-tag/SpyLigase system, and SnoopTag/SnoopCatcher systems. Additional examples of protein ligation systems are well known to a person of ordinary skill in the art and such embodiments are within the purview of the invention.

Accordingly, an antigen binding fragment is produced as a fusion protein with a C-terminally fused first binding motif of a protein ligation system and an Fc fragment is produced as a fusion protein with an N-terminally fused second binding motif of the protein ligation system. The antigen binding fragment fusion protein and the Fc fragment fusion protein can be mixed with each other (when appropriate, in the presence of a suitable enzyme in case of the enzymatic protein ligation) to produce an artificial full-length antibody containing the C-terminal first binding motif of the antigen binding fragment fusion protein covalently conjugated to the N-terminal second binding motif of the Fc fusion protein.

Any of the protein ligation systems indicated above or known in the art can be used to produce the full-length antibodies. Certain such protein ligations systems are discussed below.

### SpyTag/SpyCatcher

U.S. Pat. No. 9,547,003 (the disclosure of which is hereby incorporated by reference in its entirety) discloses the components of the SpyTag/SpyCatcher system. In this respect, peptide tags and binding partners disclosed in the U.S. Pat. No. 9,547,003 can be used as binding motifs in the instant invention. Thus, binding motifs suitable for use in the instant invention can be derived from SEQ ID NO: 1 or 3 or 5 or 6 and can be of any length, for example, about 5-50 amino acids in length (e.g., about 10, 20, 30, 40 or 50 amino acids in length) or longer. Exemplary first and second binding motifs for the SpyTag/SpyCatcher System are provided in Table 1.

The binding motifs may be fused to Fc fragment at the N-terminus or the antigen binding fragment at the C-terminus. Particularly, a spacer sequence (e.g., a glycine/serine rich spacer) may flank the binding motifs to enhance accessibility for reaction. As is apparent, the first and second binding motifs can be interchanged on one of the antibody fragments (e.g., the first binding motif can be fused to the N-terminus of a Fc fragment and the second binding motif can be fused to an antigen binding fragment at its C-terminus or the second binding motif can be fused to the N-terminus of a Fc fragment and the first binding motif can be fused to an antigen binding fragment at its C-terminus).

Thus, in certain embodiments, the first binding motif may comprise residues 302-308 of the sequence set out in SEQ ID NO: 1 or SEQ ID NO: 25 or SEQ ID NO: 27, or a sequence with at least 50% identity to SEQ ID NO: 1 or 25 or 27, wherein said first binding motif is less than 50 amino acids in length. In certain embodiments, the first binding motif has at least about 55%, about 60%, about 65%, about 70%, about 75%, about 80%, about 85, about 90, or about 95% identity to SEQ ID NO: 1 and is less than 50 amino acids in length. More particularly, the first binding motif may comprise residues 302-308, 301-308, 300-308, 299-308, 298-308, 297-308, 296-308, 295-308, 294-308, 293-308, 292-308, 291-308 or 290-308 of SEQ ID NO: 1 or a sequence with at least about 50% to 95% identity to these sequences. Preferably, the first binding motif comprises the reactive asparagine of position 303 in SEQ ID NO: 1, i.e., this residue is preferably unchanged. Further, the first binding motif may be a fragment of SEQ ID NO: 1 or 25 and, in a preferred embodiment, a first binding motif is less than 50 amino acids and comprises residues 293-308 of the sequence set forth in SEQ ID NO: 1 or comprises a sequence with at least 50% identity thereto. The first binding motif preferably contains less than 50 amino acids. Thus the first binding motif does not comprise the sequence of SEQ ID NO: 1 but only specific fragments thereof, or sequences with at least 50% identity e.g., 75, 80, 85, 90 or 95% identity to such specific fragments. Other embodiments utilize SEQ ID NO: 25 or a sequence having at least 50% sequence identity thereto as a binding motif.

If the first binding motif is any of the sequences listed in the preceding paragraph, the second binding motif can comprise or consist of residues 31-291 of the sequence set out in SEQ ID NO: 1 or SEQ ID NO: 26 or SEQ ID NO: 28 or a sequence with at least 50% identity thereto e.g., with 75, 80, 85, 90, 95, 96, 97, 98 or 99% identity to residues 32-291 of SEQ ID NO: 1 or SEQ ID NO: 26 or SEQ ID NO: 28. Specifically excluded is the complete sequence set out in SEQ ID NO: 1, however, the second binding motif preferably contains the reactive lysine corresponding to position 179 of SEQ ID NO: 1. Particularly, the second binding motif comprises residues 31-292, 31-293, 31-294, 31-295, 31-296, 31-297, 31-298, 31-299, 31-300, 31-301 or 31-302 of the sequence set forth in SEQ ID NO: 1 or a sequence with at least 70% identity thereto, excluding the sequence of SEQ ID NO: 1.

Alternatively, in certain embodiments, the second binding motif may comprise residues 302-308 of the sequence set out in SEQ ID NO: 1 or SEQ ID NO: 25 or SEQ ID NO: 27, or a sequence with at least 50% identity to residues 302-308 of SEQ ID NO: 1, SEQ ID NO: 1 or 25 or 27, wherein said second binding motif is less than 50 amino acids in length. In certain embodiments, the second binding motif has at least about 55%, about 60%, about 65%, about 70%, about 75%, about 80%, about 85, about 90, or about 95% identity to SEQ ID NO: 1 and is less than 50 amino acids in length. More particularly, the second binding motif may comprise residues 302-308, 301-308, 300-308, 299-308, 298-308, 297-308, 296-308, 295-308, 294-308, 293-308, 292-308, 291-308 or 290-308 of SEQ ID NO: 1 or a sequence with at least about 50% to 95% identity to these sequences. Preferably the second binding motif comprises the reactive asparagine of position 303 in SEQ ID NO: 1, i.e., this residue is preferably unchanged. Further, the second binding motif may be a fragment of SEQ ID NO: 1 or 25 and, in a preferred embodiment, a second binding motif is less than 50 amino acids and comprises residues 293-308of the sequence set forth in SEQ ID NO: 1 or comprises a sequence with at least 50% identity thereto. The second binding motif preferably contains less than 50 amino acids. Thus the second binding motif does not comprise the sequence of SEQ ID NO: 1 but only specific fragments thereof, or sequences with at least 50% identity e.g., 75, 80, 85, 90 or 95% identity to such specific fragments. Other embodiments utilize SEQ ID NO: 25 or a sequence having at least 50% sequence identity thereto as a binding motif.

If the second binding motif is any of the sequences listed in the preceding paragraph, the first binding motif can comprise or consist of residues 31-291 of the sequence set out in SEQ ID NO: 1 or SEQ ID NO: 26 or SEQ ID NO: 28 or a sequence with at least 50% identity thereto e.g., with 75, 80, 85, 90, 95, 96, 97, 98 or 99% identity to residues 32-291 of SEQ ID NO: 1 or SEQ ID NO: 26 or SEQ ID NO: 28. Specifically excluded is the complete sequence set out in SEQ ID NO: 1, however, the first binding motif preferably contains the reactive lysine corresponding to position 179 of SEQ ID NO: 1. Particularly, the first binding motif comprises residues 31-292, 31-293, 31-294, 31-295, 31-296, 31-297, 31-298, 31-299, 31-300, 31-301 or 31-302 of the sequence set forth in SEQ ID NO: 1 or a sequence with at least 70% identity thereto, excluding the sequence of SEQ ID NO: 1.

In specific embodiments, the first binding motif comprises SEQ ID NO: 7 or a sequence with at least 70% identity to SEQ ID NO: 7 and the second binding motif comprises SEQ ID NO: 8 or a sequence with at least 50% identity to SEQ ID NO: 8. Alternatively, the first binding motif comprises SEQ ID NO: 8 or a sequence with at least 50% identity to SEQ ID NO: 8 and the second binding motif comprises SEQ ID NO: 7 or a sequence with at least 70% identity to SEQ ID NO: 7.

In a further embodiment, the first binding motif comprises SEQ ID NO: 7 or a sequence with at least 70% identity to SEQ ID NO: 7 and the second binding motif comprises SEQ ID NO: 9 or a sequence with at least 50% identity to SEQ ID NO: 9. Alternatively, the first binding motif comprises SEQ ID NO: 9 or a sequence with at least 50% identity to SEQ ID NO: 9 and the second binding motif comprises SEQ ID NO: 7 or a sequence with at least 70% identity to SEQ ID NO: 7.

In an even further embodiment, the first binding motif comprises SEQ ID NO: 34 or a sequence with at least 70% identity to SEQ ID NO: 34 and the second binding motif comprises SEQ ID NO: 28 or a sequence with at least 50% identity to SEQ ID NO: 28. Alternatively, the first binding motif comprises SEQ ID NO: 28 or a sequence with at least 50% identity to SEQ ID NO: 28 and the second binding motif comprises SEQ ID NO: 34 or a sequence with at least 70% identity to SEQ ID NO: 34.

SpyTag002 (SEQ ID NO: 34) reacts with SpyCatcher002 (SEQ ID 28), SpyCatcher (SEQ ID NO: 8), SpyCatcher with only 84 amino acids (SpyCatcher short, SEQ ID NO: 9), and SpyCatcher003 (SEQ ID NO: 44). Therefore, in some embodiments, within one system, i.e., the CnaB2 domain from S. pyogenes, the variations of the binding motifs can be interchanged. For example, the first binding motif can be SEQ ID NO: 34 or a sequence with at least 70% identity to SEQ ID NO: 34 and the second binding motif comprises SEQ ID NO: 28, 8, 9 or 44 or a sequence with at least 50% identity to SEQ ID NO: 28, 8, 9 or 44. Alternatively, the first binding motif can be SEQ ID NO: 28, 8, 9 or 44 or a sequence with at least 50% identity to SEQ ID NO: 28, 8, 9 or 44 and the second binding motif comprises SEQ ID NO: 34 or a sequence with at least 70% identity to SEQ ID NO: 34.

In a further embodiment, the first binding motif comprises SEQ ID NO: 43 (SpyTag003) or a sequence with at least 70% identity to SEQ ID NO: 43 and the second binding motif comprises SEQ ID NO: 44 (SpyCatcher003) or a sequence with at least 50% identity to SEQ ID NO: 44. Alternatively, the first binding motif comprises SEQ ID NO: 44 or a sequence with at least 50% identity to SEQ ID NO: 44 and the second binding motif comprises SEQ ID NO: 43 or a sequence with at least 70% identity to SEQ ID NO: 43.

Additionally, a binding motif may be designed from the major pilin protein Spy0128 using the alternative isopeptide bond in the N-terminus. Spy0128 is a major pilin protein Spy0128, which has an amino acid sequence as set out in SEQ ID NO: 1 and is encoded by a nucleotide sequence as set out in SEQ ID NO: 2. Use of Spy0128 for protein ligation is described by Abe et al. (2013), Bioconjugate Chem., 24 (2): 242-250. The Abe et al. reference is incorporated by reference in its entirety. Thus, a binding motif may be designed or is obtainable from an N-terminal fragment of the isopeptide protein and the remaining, truncated or overlapping protein fragment may constitute the other binding motif. The reactive lysine involved in the isopeptide bond at the N-terminus is found at position 36 of SEQ ID NO: 1 and the reactive asparagine involved in the isopeptide bond is found at position 168 of SEQ ID NO: 1.

Thus, in a preferred embodiment, one of the binding motifs comprises the reactive lysine residue and the other binding motif comprises a reactive glutamic acid, aspartic acid or asparagine. Particularly, in certain embodiments, a first binding motif comprises residues 31-4 0of the sequence set out in SEQ ID NO: 1 or a sequence with at least 70% identity thereto and is less than 50 amino acids in length; whereas, the second binding motif comprises residues 37-304 of the sequence set out in SEQ ID NO: 1 or has a sequence with at least 70% identity thereto, excluding the sequence of SEQ ID NO: 1. Alternatively, in certain embodiments, a second binding motif comprises residues 31-40 of the sequence set out in SEQ ID NO: 1 or a sequence with at least 70% identity thereto and is less than 50 amino acids in length; whereas, the first binding motif comprises residues 37-304 of the sequence set out in SEQ ID NO: 1 or has a sequence with at least 70% identity thereto, excluding the sequence of SEQ ID NO: 1. Preferably, the reactive residues in the binding motifs are not mutated.

In further embodiments, a first binding motif comprises residues 179-184 e.g., 173-185 of the sequence set out in SEQ ID NO: 3 or has a sequence with at least 50% identity thereto and is less than 50 amino acids in length. In such embodiments, the second binding motif comprises residues 191-317 e.g., 186-318 of SEQ ID NO: 3 or a sequence having at least 50% identity thereto, excluding SEQ ID NO: 3. Alternatively, in even further embodiments, a second binding motif comprises residues 179-184 e.g., 173-185 of the sequence set out in SEQ ID NO: 3 or has a sequence with at least 50% identity thereto and is less than 50 amino acids in length. In such embodiments, the first binding motif comprises residues 191-317 e.g., 186-318 of SEQ ID NO: 3 or a sequence having at least 50% identity thereto, excluding SEQ ID NO: 3. Specifically excluded as a binding motif is the full-length sequence of SEQ ID NO: 3.

In even further embodiments, a first binding motif comprises a fragment of SEQ ID NO: 5 that includes the asparagine at position 266 (or sequences having at least 50% identity thereto) and a second binding motif comprises a fragment of SEQ ID NO: 5 or a sequence having at least 50% sequence identity thereto and that comprises the lysine residue at position 149 but does not include the asparagine at position 266. Alternatively, in even further embodiments, a second binding motif comprises a fragment of SEQ ID NO: 5 that includes the asparagine at position 266 (or sequences having at least 50% identity thereto) and a first binding motif comprises a fragment of SEQ ID NO: 5 or a sequence having at least 50% sequence identity thereto and that comprises the lysine residue at position 149 but does not include the asparagine at position 266. Preferably, none of the binding motifs comprises SEQ ID NO: 5.

In yet additional embodiments, a first binding motif comprises a fragment of SEQ ID NO: 6 that includes the aspartic acid residue at position 101 (or a sequence at least 70% identical thereto) and a second binding motif comprises a fragment of SEQ ID NO: 6 that contains the reactive lysine of position 15 (or sequences at least 50% identical thereto). Alternatively, in additional embodiments, a second binding motif comprises a fragment of SEQ ID NO: 6 that includes the aspartic acid residue at position 101 (or a sequence at least 70% identical thereto) and a first binding motif comprises a fragment of SEQ ID NO: 6 that contains the reactive lysine of position 15 (or sequences at least 50% identical thereto). None of these binding motifs comprises SEQ ID NO: 6.

Another embodiment provides for a first binding motif comprising SEQ ID NO: 25 or SEQ ID NO: 27, or a sequence with at least 50% identity to SEQ ID NO: 25 or 27, wherein said binding motif is 15 to 40 or 50 amino acids in length. In certain embodiments, the first binding motif has at least about 55%, about 60%, about 65%, about 70%, about 75%, about 80%, about 85, about 90, or about 95% identity to SEQ ID NO: 25 or 27 and is less than 50 amino acids in length. Preferably a first binding motif comprises the reactive aspartic acid of position 8 in SEQ ID NO: 27, i.e., this residue is preferably unchanged.

If the first binding motif is any of the sequences listed in the preceding paragraph, a second binding motif comprises or consists of SEQ ID NO: 26 or a sequence with at least 50% identity thereto e.g., with 75, 80, 85, 90, 95, 96, 97, 98 or 99% identity to SEQ ID NO: 26 or SEQ ID NO: 28 or a sequence with at least 50% identity thereto, e.g., with 75, 80, 85, 90, 95, 96, 97, 98 or 99% identity to SEQ ID NO: 28. Variants have at least 50% sequence identity and retain the lysine at position 57 of SEQ ID NO: 26.

Additional embodiments provide for a second binding motif comprising SEQ ID NO: 25or SEQ ID NO: 27, or a sequence with at least 50% identity to SEQ ID NO: 25 or 27, wherein said binding motif is 15 to 40 or 50 amino acids in length. In certain embodiments, a second binding motif has at least about 55%, about 60%, about 65%, about 70%, about 75%, about 80%, about 85, about 90, or about 95% identity to SEQ ID NO: 25 or 27 and is less than 50 amino acids in length. Preferably a second binding motif comprises the reactive aspartic acid of position 8 in SEQ ID NO: 27, i.e., this residue is preferably unchanged.

If the second binding motif is any of the sequences listed in the preceding paragraph, a first binding motif comprises or consists of SEQ ID NO: 26 or a sequence with at least 50% identity thereto e.g., with 75, 80, 85, 90, 95, 96, 97, 98 or 99% identity to SEQ ID NO: 26 or SEQ ID NO: 28 or a sequence with at least 50% identity thereto, e.g., with 75, 80, 85, 90, 95, 96, 97, 98 or 99% identity to SEQ ID NO: 28. Variants have at least 50% sequence identity and retain the lysine at position 57 of SEQ ID NO: 26.

In specific embodiments, a first binding motif comprises SEQ ID NO: 39 or a sequence with at least 70% identity thereto; whereas, the second binding motif comprises SEQ ID NO: 40 or a sequence with at least 50% identity thereto. Alternatively, a first binding motif comprises SEQ ID NO: 40 or a sequence with at least 50% identity thereto; whereas, the second binding motif comprises SEQ ID NO: 39 or a sequence with at least 70% identity thereto.

### SpyLigase/SnoopLigase

Alternatively, an Fc fragment can be conjugated to an antigen binding fragment using the systems described in WO2016/193746 and Veggiani et al., 2016 (each of which is hereby incorporated by reference in its entirety). In such embodiments, binding motifs are attached the Fc fragments and antigen binding fragments, optionally through linker sequences, such as a glycine/serine rich spacer. These binding motifs are then ligated by a ligase. Each of the first and the second binding motifs can, in some embodiments, have a length between 6-50 amino acids, e.g., 7-45, 8-40, 9-35, 10-30, 11-25 amino acids in length, e.g., it may comprise or consist of 6, 7, 8, 9, 10, 11, 12, 13, 14, 15, 16, 17, 18, 19 or 20 amino acids. In other embodiments, each of the first and the second binding motifs is about 20-300 amino acids in length (e.g., about 10, 20, 30, 40, 50, 60, 70, etc. amino acids in length). In some embodiments, the peptide ligase may be between 50-300 amino acids in length, e.g., 60-250, 70-225, 80-200 amino acids in length, e.g., it may comprise or consist of 60, 65, 70, 75, 80, 85, 90, 95, 100, 110, 120, 130, 140, 150, 160, 170, 180, 190 or 200 amino acids, providing it meets the definitions set forth for the ligase, below.

In certain embodiments, the SpyLigase system of protein ligation is used. In such embodiments, the antigen binding fragment-SpyTag fusion protein can be produced as described above. The Fc fragment is not fused to the SpyCatcher, but is fused to the 10 amino acid K-Tag (ATHIKFSKRD, SEQ ID NO: 33), at its N-terminus and optionally, with the one or more linkers as described above. The purified antigen binding fragment-SpyTag and the purified K-Tag-Fc fragment fusion protein can be mixed in the presence of SpyLigase to form the covalent bonds between the two molecules. Thus, in certain such embodiments, the first binding motif comprising a SpyTag is present at the C-terminus of an antigen binding fragment and a second binding motif comprising a K-Tag (SEQ ID NO: 33) is present at the N-terminus of an Fc fragment. Alternatively, the first binding motif comprising a K-Tag (SEQ ID NO: 33) is present at the C-terminus of an antigen binding fragment and a second binding motif comprising a SpyTag is present at the N-terminus of an Fc fragment.

The advantage of these embodiments is the shorter K-tag compared to the SpyCatcher, leading to an antibody that resembles the natural antibody. For example, if the natural “hinge-linker” is used only 23 “non-natural” amino acids would be present between a Fab portion and the Fc portion in an artificial full-length antibody.

### Snoop Tag.Jr/DogTag/SnoopLigase

Additionally, a binding motif may be designed from RrgA protein of S. pneumoniae as described by Buldun et al. (2018). The Buldun et al. reference is incorporated by reference in its entirety. Thus, in certain embodiments, a first binding motif comprises SEQ ID NO: 37 or a sequence with at least 70% identity thereto; whereas, the second binding motif comprises SEQ ID NO: 38 or a sequence with at least 70% identity thereto. Alternatively, a first binding motif comprises SEQ ID NO: 38 or a sequence with at least 70% identity thereto; whereas, the second binding motif comprises SEQ ID NO: 37 or a sequence with at least 70% identity thereto. A SnoopLigase can be provided which facilitates the formation of the bond between the first and the second binding motifs.

In certain embodiments, a pair of first and the second binding motifs may be derived from any suitable isopeptide protein. For instance, each of the first and the second binding motifs may be derived from the major pilin protein Spy0128, which has an amino acid sequence as set out in SEQ ID NO: 1 and is encoded by a nucleotide sequence as set out in SEQ ID NO: 2. Two isopeptide bonds are formed in the protein. One isopeptide bond is formed between lysine at position 179 in SEQ ID NO: 1 and asparagine at position 303 in SEQ ID NO: 1 (the reactive residues). The glutamic acid residue which induces the spontaneous isopeptide bond is found at position 258 in SEQ ID NO: 1. Thus, a pair of binding motifs developed from an isopeptide protein set forth in SEQ ID NO: 1 will preferably comprise a first binding motif comprising a fragment of the protein comprising the reactive asparagine at position 303 and a second binding motif comprising a fragment of the protein comprising the reactive lysine at position 179. Alternatively, a pair of binding motifs developed from an isopeptide protein set forth in SEQ ID NO: 1 will preferably comprise a second binding motif comprising a fragment of the protein comprising the reactive asparagine at position 303 and a first binding motif comprising a fragment of the protein comprising the reactive lysine at position 179. In such embodiments, a fragment of the protein comprising the glutamic acid residue at position 258 can be provided separately, i.e., as a peptide ligase that forms the isopeptide bond.

Another isopeptide bond in the major pilin protein Spy0128 occurs between the lysine residue at position 36 of SEQ ID NO: 1 and the asparagine residue at position 168 of SEQ ID NO: 1. The glutamic acid residue which induces isopeptide formation is found at position 117 in SEQ ID NO: 1. Thus, a pair of binding motifs developed from an isopeptide protein set forth in SEQ ID NO: 1 will preferably comprise a first binding motif comprising a fragment of the protein comprising the reactive lysine residue at position 36 and a second binding motif comprising a fragment of the protein comprising the reactive asparagine at position 168. Alternatively, a pair of binding motifs developed from an isopeptide protein set forth in SEQ ID NO: 1 will preferably comprise a second binding motif comprising a fragment of the protein comprising the reactive lysine residue at position 36 and a first binding motif comprising a fragment of the protein comprising the reactive asparagine at position 168. In such embodiments, a fragment of the protein comprising the glutamic acid residue at position 117 may be provided separately as a peptide ligase.

An isopeptide bond occurs between a lysine residue at position 181 of SEQ ID NO: 3 (ACE19, a domain of an adhesin protein from E. faecalis) and an asparagine residue at position 294 of SEQ ID NO: 3. The bond is induced by an aspartic acid residue at position 213 in SEQ ID NO: 3. Thus, a pair of binding motifs developed from isopeptide protein set forth in SEQ ID NO: 3 will preferably comprise a first binding motif comprising a fragment of the protein comprising the reactive asparagine residue at position 294 and a second binding motif comprising a fragment of the protein comprising the reactive lysine residue at position 181. Alternatively, a pair of binding motifs developed from isopeptide protein set forth in SEQ ID NO: 3 will preferably comprise a second binding motif comprising a fragment of the protein comprising the reactive asparagine residue at position 294 and a first binding motif comprising a fragment of the protein comprising the reactive lysine residue at position 181. In such embodiments, a fragment of the protein comprising the aspartic acid residue at position 213 may be provided separately as a peptide ligase.

The collagen binding domain from S. aureus which has an amino acid sequence set out in SEQ ID NO: 10 can also be used. The isopeptide bond occurs between lysine at position 176 of SEQ ID NO: 10 and asparagine at position 308 of SEQ ID NO: 10. The aspartic acid residue which induces the isopeptide bond is at position 209 of SEQ ID NO: 10. Thus, a pair of binding motifs developed from the isopeptide protein set forth in SEQ ID NO: 10 will preferably comprise a first binding motif comprising a fragment of the protein comprising the reactive lysine at position 176 and a second binding motif comprising a fragment of the protein comprising the reactive asparagine at position 308. Alternatively, a pair of binding motifs developed from the isopeptide protein set forth in SEQ ID NO: 10 will preferably comprise a second binding motif comprising a fragment of the protein comprising the reactive lysine at position 176 and a first binding motif comprising a fragment of the protein comprising the reactive asparagine at position 308. In such embodiments, a fragment of the protein comprising the aspartic acid residue at position 209 may be provided separately as a peptide ligase.

FbaB from Streptococcus pyogenes can also be used to provide binding motifs and comprises a domain, CnaB2, which has an amino acid sequence set out in SEQ ID NO: 11, is encoded by the nucleotide sequence set out in SEQ ID NO: 12. The isopeptide bond in the CnaB2 domain forms between a lysine at position 15 of SEQ ID NO: 11 and an aspartic acid residue at position 101 of SEQ ID NO: 11. The glutamic acid residue which induces the isopeptide bond is at position 61 of SEQ ID NO: 11. Thus, a pair of binding motifs developed from the isopeptide protein set forth in SEQ ID NO: 11 will preferably comprise a first binding motif comprising a fragment of the protein comprising the reactive lysine at position 15 and a second binding motif comprising a fragment of the protein comprising the reactive aspartic acid at position 101. Alternatively, a pair of binding motifs developed from the isopeptide protein set forth in SEQ ID NO: 11 will preferably comprise a second binding motif comprising a fragment of the protein comprising the reactive lysine at position 15 and a first binding motif comprising a fragment of the protein comprising the reactive aspartic acid at position 101. In such embodiments, a fragment of the protein comprising the glutamic acid residue at position 61 may be provided separately as a peptide ligase.

The RrgA protein is an adhesion protein from Streptococcus pneumoniae, which has an amino acid sequence as set out in SEQ ID NO: 13 and is encoded by a nucleotide sequence as set out in SEQ ID NO: 14. An isopeptide bond is formed between lysine at position 742 in SEQ ID NO: 13 and asparagine at position 854 in SEQ ID NO: 13. The bond is induced by a glutamic acid residue at position 803 in SEQ ID NO: 13. Thus, a pair of binding motifs developed from the isopeptide protein set forth in SEQ ID NO: 13 will preferably comprise a first binding motif comprising a fragment of the protein comprising the reactive asparagine at position 854 and a second binding motif comprising a fragment of the protein comprising the reactive lysine at position 742. Alternatively, a pair of binding motifs developed from the isopeptide protein set forth in SEQ ID NO: 13 will preferably comprise a second binding motif comprising a fragment of the protein comprising the reactive asparagine at position 854 and a first binding motif comprising a fragment of the protein comprising the reactive lysine at position 742. In such embodiment, a fragment of the protein comprising the glutamic acid residue at position 803 may be provided separately as a peptide ligase as defined above.

The PsCs protein is a fragment of the por secretion system C-terminal sorting domain protein from Streptococcus intermedius, which has an amino acid sequence as set out in SEQ ID NO: 15 and is encoded by a nucleotide sequence as set out in SEQ ID NO: 16. An isopeptide bond is formed between lysine at position 405 in SEQ ID NO: 15 and aspartate at position 496 in SEQ ID NO: 15. Thus, a pair of binding motifs developed from the isopeptide protein set forth in SEQ ID NO: 15 will preferably comprise a first binding motif comprising a fragment of the protein comprising the reactive aspartate at position 496 and a second binding motif comprising a fragment of the protein comprising the reactive lysine at position 405. Alternatively, a pair of binding motifs developed from the isopeptide protein set forth in SEQ ID NO: 15 will preferably comprise a first binding motif comprising a fragment of the protein comprising the reactive aspartate at position 496 and a second binding motif comprising a fragment of the protein comprising the reactive lysine at position 405.

In various embodiments, the first and the second binding motifs may be derived from an isopeptide protein comprising an amino acid sequence as set forth in any one of SEQ ID NO: 21 or 23 or 25 or 27 or a protein with at least 70% sequence identity to an amino acid sequence as set forth in any one of SEQ ID NO: 21 or 23 or 25 or 27. In some embodiments, said isopeptide protein sequence above is at least 75, 80, 85, 90, 95, 96, 97, 98, 99 or 100% identical to the sequence (SEQ ID NO: 21 or 23 or 25 or 27) to which it is compared.

### SnoopTagJr/DogTag/SnoopLigase

Additionally, a binding motif may be designed from RrgA protein of S. pneumoniae as described by Buldun et al. (2018). The Buldun et al. reference is incorporated by reference in its entirety. Thus, in certain embodiments, a first binding motif comprises SEQ ID NO: 37 or a sequence with at least 70% identity thereto; whereas, the second binding motif comprises SEQ

ID NO: 38 or a sequence with at least 70% identity thereto. Alternatively, a first binding motif comprises SEQ ID NO: 38 or a sequence with at least 70% identity thereto; whereas, the second binding motif comprises SEQ ID NO: 37 or a sequence with at least 70% identity thereto. A SnoopLigase can be provided which facilitates the formation of the bond between the first and the second binding motifs.

### SdyTag/SdyCatcher (DANG Short)

In further embodiments, a binding motif may be designed from fibronectin binding protein of S. dysgalactiae. Such protein ligation is described by Tan et al. (2016). The Tan et al. reference is incorporated by reference in its entirety. Thus, in certain embodiments, a first binding motif comprises SEQ ID NO: 41 or a sequence with at least 70% identity thereto; whereas, the second binding motif comprises SEQ ID NO: 42 or a sequence with at least 50% identity thereto. Alternatively, a first binding motif comprises SEQ ID NO: 42 or a sequence with at least 50% identity thereto; whereas, the second binding motif comprises SEQ ID NO: 41 or a sequence with at least 70% identity thereto.

### Sortase

Another means for conjugating an Fc fragment to an antigen binding fragment comprises the use of sortase enzymes and sortase recognition and bridging domains. Schmohl et al. (2014), which is hereby incorporated by reference in its entirety, discuss sortase mediated ligation for the site-specific modification of proteins. In this aspect of the invention, the sortase recognition and bridging domains are considered binding motifs. Sortases are transpeptidases produced by Gram-positive bacteria to anchor cell surface proteins covalently to the cell wall. The Staphylococcus aureus sortase A (SrtA) cleaves a short C-terminal recognition motif (LPXTG (SEQ ID NO: 17) (referred to herein as a sortase recognition domain). The sortase recognition domain is a sortase A recognition domain or a sortase B recognition domain. In particular embodiments, the sortase recognition domain comprises or consists of the amino acid sequence: LPTGAA (SEQ ID NO: 18), LPTGGG (SEQ ID NO: 19), LPKTGG (SEQ ID NO: 20), LPETG (SEQ ID NO: 21), LPXTG (SEQ ID NO: 22) or LPXTG (X)ₙ (SEQ ID NO: 23), where X is any amino acid, and n is 0, 1, 2, 3, 4, 5, 7, 8, 9, 10, in the range of 0-5 or 0-10, or any integer up to 100.

The sortase A bridging domain comprises one or more glycine residues at one of its termini. In certain embodiments, the one or more glycine residues may optionally be: Gly, (Gly)₂, (Gly)₃, (Gly)₄, or (Gly) x, where x is an integer of 1-20. The sortase A recognition domain can be fused to an antigen binding fragment at the C-terminus, optionally, through a glycine/serine rich spacer, and sortase A bridging domain can be fused to an Fc fragment at the N-terminus, optionally, through a glycine/serine rich spacer.

Thus, in certain embodiments, a first binding domain fused to an antigen binding fragment at the C-terminus comprises a sortase A recognition domain comprising or consisting of the amino acid sequence: LPTGAA (SEQ ID NO: 18), LPTGGG (SEQ ID NO: 19), LPKTGG (SEQ ID NO: 20), LPETG (SEQ ID NO: 21), LPXTG (SEQ ID NO: 22) or LPXTG (X)ₙ (SEQ ID NO: 23), where X is any amino acid, and n is 0, 1, 2, 3, 4, 5, 7, 8, 9, 10, in the range of 0-5 or 0-10, or any integer up to 100 and the second binding domain fused to an Fc fragment at the N-terminus comprises a sortase A bridging domain comprising or consisting of: Gly, (Gly)₂, (Gly)₃, (Gly)₄, or (Gly)ₓ, where x is an integer of 1-20.

The sortase B recognition domain comprises the amino acid sequence NPX1TX2 (SEQ ID NO: 24), where X1 is glutamine or lysine; X2 is asparagine or glycine; N is asparagine; P is proline and T is threonine. The sortase B bridging domain comprises one or more glycine residues at one of its termini. In certain embodiments, the one or more glycine residues may optionally be: Gly, (Gly)₂, (Gly)₃, (Gly)₄, or (Gly)ₓ, where x is an integer of 1-20. The sortase B recognition domain can be fused to an antigen binding fragment at the C-terminus, optionally, through a glycine/serine rich spacer, and sortase B bridging domain can be fused to an Fc fragment at the N-terminus, optionally, through a glycine/serine rich spacer.

Thus, in certain embodiments, a first binding domain fused to an antigen binding fragment at the C-terminus comprises a sortase recognition domain comprising or consisting of the amino acid sequence: NPX1TX2 (SEQ ID NO: 24), where X1 is glutamine or lysine; X2 is asparagine or glycine; N is asparagine; P is proline and T is threonine and the second binding domain fused to an Fc fragment at the N-terminus comprises a sortase B bridging domain comprising or consisting of: Gly, (Gly)₂, (Gly)₃, (Gly)₄, or (Gly)ₓ, where x is an integer of 1-20.

### Butelase

Yet another means for conjugating an Fc fragment to an antigen binding fragment comprises the use of butelase 1 to form a peptide bond between the butelase recognition motif (where Asx is Asn or Asp) and the amino terminus of another polypeptide. In this case, the Asx-His-Val motif can be fused, in frame, to an antigen binding fragment, optionally through a glycine/serine rich spacer. Butelase can then be used to form a peptide bond between the Asx-His-Val motif and the N-terminal amino acid of the Fc fragment. WO 2017/058114, which is hereby incorporated by reference in its entirety, discloses methods and materials for butelase-mediated peptide ligation.

Thus, in certain embodiments, a first binding domain fused to an antigen binding fragment at the C-terminus comprises a butelase recognition domain comprising or consisting of the amino acid sequence: Asx-His-Val (where Asx is Asn or Asp), and the second binding domain comprises the N-terminal amino acid of the Fc fragment.

### Split Inteins

Another method for conjugating an Fc fragment to an antigen binding fragment comprises the use of split inteins. Inteins can exist as two fragments encoded by two separately transcribed and translated genes. These so-called split inteins self-associate and catalyze protein-splicing activity in trans. Split inteins have been identified in diverse cyanobacteria and archaea (Caspi et al., 2003; Choi et al., 2006; Dassa et al., 2007; Liu and Yang, 2003; Wu et al., 1998; and Zettler et al., 2009, the disclosures of which are hereby incorporated by reference in their entireties). Thiel et al. (2014) and WO 2013/045632, each of which is hereby also incorporated by reference in its entirety, also disclose the use of split inteins that can be used to fuse an antigen binding fragment and an Fc fragment.

Thus, in certain embodiments, a first binding domain comprises a first split intein and a second binding domain comprises a second split intein, wherein the first split intein and the second split intein bind to form a catalytically competent enzyme. Then they catalyze their own excision and the ligation of their flanking sequences.

Any of the protein ligation systems described above or otherwise known in the art can be used to design binding motifs that are fused to the antigen binding fragments and Fc fragments to produce full-length antibodies of the invention.

For example, in certain embodiments, an antigen binding fragment is produced as a fusion protein with a C-terminally fused SpyTag as a first binding motif and an Fc fragment is produced as a fusion protein with an N-terminally fused SpyCatcher as a second binding motif. The antigen binding fragment-first binding motif fusion protein and the Fc fragment-second binding motif fusion protein can be mixed with each other to produce an artificial full-length antibody containing the antigen binding fragment with the C-terminal SpyTag conjugated to the N-terminal SpyCatcher of the Fc fragment fusion protein.

An antigen binding fragment-SpyTag fusion protein can be created by expressing the gene encoding the antigen binding fragment, for example, in Escherichia coli using a vector that adds a SpyTag to the C-terminus of the antigen binding fragment. A second tag, e.g., the His-tag, can be added before or after the SpyTag for purification of the antigen binding fragment-SpyTag fusion protein via affinity chromatography. In certain embodiments, SpyTag has a sequence of AHIVMVDAYKPTK (SEQ ID NO: 29) or AHIVMVDAYK (SEQ ID NO: 30).

The SpyCatcher-Fc fusion protein can be created separately by expressing an Fc fragment, for example, the human IgG1 Fc fragment, in a mammalian expression host cell, for example, CHO cell line or HEK293 cell line. In the vector used to express the Fc, the gene fragment encoding the heavy chain Fc (CH2-CH3), with or without the hinge region connecting CH1 and CH2 in the natural antibody, is preceded by the SpyCatcher domain, either the 116 amino acid domain (SEQ ID NO: 8), see Li et al., 2014 or the shortened 84 amino acid (SEQ ID NO: 9) version (Li et al., 2014). The region between SpyCatcher and CH2-CH3 domain may contain a peptide acting as a spacer between SpyCatcher and Fc fragment to provide additional flexibility. For example, such spacer peptide can be the natural antibody hinge region (e.g., human IgG1: EPKSCDKTHTCPPCP (SEQ ID NO: 31) or a linker peptide, for instance peptides containing one or more of the 5 amino acid GGGGS (SEQ ID NO: 32) sequence motif that is known to be both flexible and soluble, or a combination thereof. The Fc fragment fusion protein construct is preceded by a signal sequence that enables transport of the resulting Fc fragment fusion proteins outside the cell. The SpyCatcher-Fc fusion protein can be purified by standard affinity chromatography, e.g., using Protein A. Mixing an antigen binding fragment-SpyTag fusion protein with a SpyCatcher-Fc fusion protein in appropriate stoichiometry can be used to create the artificial full-length antibody. For example, a Fab-SpyTag and the SpyCatcher-Fc fusion can be mixed in the stoichiometry of 2 Fab-SpyTag molecules per SpyCatcher-Fc molecule to create full-length artificial antibodies. Other stoichiometries can be used as well to increase the product yield, for instance an excess of Fab-SpyTag. A subsequent purification step can be added to remove any surplus reactants.

Appropriate conditions, such as buffer conditions, pH, temperature and presence of detergents can be provided for optimal conjugation via SpyTag/SpyCatcher system. In one embodiment, the reaction halftime with each partner at 10 μM at 25° C. and pH 7.0 was determined to be 74 sec (Zakeri et al., 2012). The artificial full-length antibody so produced can be used as is or further purified before use. Such purification can be performed by size exclusion chromatography or affinity chromatography with an immobilized antibody that specifically binds to the complete FbaB domain but does not bind to the SpyTag or the SpyCatcher.

In a specific embodiment, conjugation is performed in the presence of an excess of Fab-SpyTag to drive the reaction towards the formation of full-length antibodies. The resulting full length antibody is then purified to remove excess Fab-SpyTag, for example, using a Protein A binding matrix or another binding interaction, such as His-tag or Strep-tag™. Such tags can be introduced into the Fc, for instance at the Fc C-terminus.

Certain Fab frameworks, for example, Fab containing VH from the VH3 germline class, also bind to Protein A. Therefore, an Fc-SpyCatcher fusion comprising a C-terminal purification tag (such as His-Tag or Strep-tag) can be used for purification to avoid contamination with Protein A binding Fab fragments.

Alternatively, an antigen binding fragment can be produced as a fusion protein with a C-terminally fused SpyCatcher as a first binding motif and an Fc fragment can be produced as a fusion protein with an N-terminally fused SpyTag as a second binding motif. The antigen binding fragment-first binding motif fusion protein and the Fc fragment-second binding motif fusion protein can be mixed with each other to produce an artificial full-length antibody containing the antigen binding fragment with the C-terminal SpyCatcher conjugated to the N-terminal SpyTag of the Fc fragment fusion protein. The sequences of SpyTags and SpyCatchers as well as the linker sequences derived from the hinge or other flexible and soluble sequence motifs discussed above can also be included in these embodiments.

In certain embodiments, the SpyLigase system of protein ligation is used. In such embodiments, the antigen binding fragment-SpyTag fusion protein can be produced as described above. The Fc fragment is not fused to the SpyCatcher, but is fused to the 10 amino acid K-Tag (ATHIKFSKRD, SEQ ID NO: 33), at its N-terminus and optionally, with the one or more linkers as described above. The purified antigen binding fragment-SpyTag and the purified K-Tag-Fc fragment fusion protein can be mixed in the presence of SpyLigase to form the covalent bonds between the two molecules. Thus, in certain such embodiments, the first binding motif comprising a SpyTag is present at the C-terminus of an antigen binding fragment and a second binding motif comprising a K-Tag (SEQ ID NO: 33) is present at the N-terminus of an Fc fragment. Alternatively, the first binding motif comprising a K-Tag (SEQ ID NO: 33) is present at the C-terminus of an antigen binding fragment and a second binding motif comprising a SpyTag is present at the N-terminus of an Fc fragment.

The advantage of these embodiments is the shorter K-tag compared to the SpyCatcher, leading to an antibody that resembles the natural antibody. For example, if the natural “hinge-linker” is used only 23 “non-natural” amino acids would be present between a Fab portion and the Fc portion in an artificial full-length antibody.

In further embodiments, instead of the SpyTag/SpyCatcher system the SnoopTag/SnoopCatcher system (Veggiani et al., 2016) is used. The SnoopTag/SnoopCatcher system follows the same principle as the SpyTag/SpyCatcher system, except the D4 Ig-like domain of the adhesin RrgA from Streptococcus pneumoniae is used as the starting point.

Furthermore, SpyTag and the SnoopTag system can be combined to produce multispecific antibodies. For example, a polymer of two or more Fc fragments can be produced where each of the Fc fragments has at its N-terminus a specific second binding motif, wherein one or more second binding motifs in the polymer of two or more Fc fragments are from the SpyTag system and one or more second binding motifs in the polymer of two or more Fc fragments are from the SnoopTag system. Such a polymer of two or more Fc fragments can be contacted with a plurality of antigen-binding fragments, each specific for a different antigen or epitope, and each having a specific first binding motif, wherein one or more first binding motifs in the one or more antigen-binding fragments from the plurality of antigen-binding fragments are from the SpyTag system and one or more first binding motifs in the one or more antigen-binding fragments from the plurality of antigen-binding fragments are from the SnoopTag system.

Polymers of two or more Fc fragments can be produced by techniques known in the art. Certain such techniques are described by Mekhaiel et al. (2011), Scientific Reports; 1:124 and Czajkowsky et al. (2012), EMBO Mol. Med.; 4(10): 1015-1028. Additional techniques for producing polymers having two or more Fc fragments are known in the art and such embodiments are within the purview of the invention.

In even further embodiments, the Sortase system is used to create the full-length artificial antibody. In such embodiments, an antigen binding fragment contains the sorting motif LPXTG (SEQ ID NO: 17) at the C-terminus, for example, C-terminus of the heavy chain of a Fab fragment. The Fc part is produced with a GG sequence at the N-terminus to allow covalent coupling by the addition of Sortase. Sortase mediated conjugation reaction can be performed in a Ca²⁺ containing buffer. In these embodiments, the number of “non-natural” amino acids between the Fab and the Fc fragment is only six, if the “hinge-linker” is used as a spacer. Alternatively, an antigen binding fragment contains a GG sequence at the C-terminus and the sorting motif LPXTG (SEQ ID NO: 17) is present at the N-terminus of the Fc fragment. The covalent coupling is catalyzed by Sortase.

In further embodiments, the Split Inteins system (Shah et al., 2011) or the Butelase mediated ligation (Nguyen et al., 2016) is used. In these systems, an enzyme formed from the two components of the Split Inteins system catalyzes covalent bond formation between the antigen binding fragment fusion protein and the Fc fragment fusion protein. As such, in various embodiments of the invention, a series of Fc fragment-fusion proteins, all equipped with a binding motif at the N-terminus that allows site-specific covalent protein conjugation, either by autocatalysis (SpyTag and SnoopTag systems, Split Inteins) or by enzyme-mediated catalysis (Sortase, SpyLigase, SnoopLigase, Butelase) are produced. Antigen binding fragments, for instance derived from library technologies are produced with the corresponding binding motif at the C-terminus. Such fragments contribute the specificities needed for the assay. Any of such antibody fragments can be combined with any of the prepared Fc fragment-fusion proteins to create full-length antibodies that contain both the specificity to a target and the Fc part needed to allow assay multiplexing or usage as control or calibrator in a diagnostic assay that measures patient antibody titer to a given antigen.

### Multiplex Assays

The plurality of full-length antibodies provided herein can be used to identify a plurality of antigens in the same reaction. Therefore, certain embodiments of the invention provide a method of determining the levels of a plurality of antigens in a sample, comprising contacting the sample with a plurality of full-length antibodies provided herein, and quantifying the binding between each of the plurality of full-length antibodies and their corresponding antigens to determine the levels of each of the plurality of antigens in the sample.

Various methods of visualizing and quantifying the binding between antibodies and their corresponding antigens are known in the art and such embodiments are within the purview of the invention. For example, a combination of unique beads is provided by the Bio-Plex® multiplex immunoassay system can be used to quantify the binding between each of the plurality of full-length antibodies and their corresponding antigens. Alternatively, a combination of unique labels can be used to quantify the binding between each of the plurality of full-length antibodies and their corresponding antigens.

### Nucleic Acid Constructs

Further embodiments of the invention provide nucleic acid constructs that encode for the antigen binding fragments fused with certain binding motifs as well as Fc fragments fused with the corresponding binding motifs. Such nucleic acids can be present in an expression vector in an appropriate host cell. As discussed below, the host cells can be prokaryotic or eukaryotic.

Accordingly, certain embodiments of the invention provide a plurality of pairs of nucleic acid constructs, wherein each pair of nucleic acid construct comprises:
a) a first nucleic acid construct comprising a polynucleotide sequence encoding an antigen binding fragment fused at the C-terminus to a first binding motif; and
b) a second nucleic acid construct comprising a polynucleotide encoding an Fc fragment fused at the N-terminus to a second binding motif,
wherein each antigen binding fragment specifically binds to a unique antigen and each Fc fragment belongs to a unique combination of species, isotype and subclass, and
wherein the first binding motif and the second binding motif form a covalent bond when brought into contact with one another either spontaneously or with the help of an enzyme.

Typically, a polynucleotide sequence encoding a Fab fused at the C-terminus to a first binding motif encodes two peptides, namely, L and H chain of the Fab. A first binding motif, such as a SpyTag, can be fused to either L or H chain. Preferably, a first binding motif is fused to the H chain. A Fab expression cassette can comprise a bicistronic vector that produces one mRNA encoding both L and H chains. Also, both H and L chains have a signal peptide to direct their export into the periplasm.

In certain embodiments, the invention provides a plurality of nucleic acid constructs, wherein each nucleic acid construct comprises a polynucleotide sequence encoding an antigen binding fragment fused at the C-terminus to a first binding motif, wherein each antigen binding fragment specifically binds to a unique antigen, and wherein the first binding motif forms a covalent bond with a second binding motif when brought into contact with one another either spontaneously or with the help of an enzyme.

Further embodiments of the invention provide a plurality of nucleic acid constructs, wherein each nucleic acid construct comprises a polynucleotide encoding an Fc fragment fused at the N-terminus to a second binding motif, wherein each Fc fragment belongs to a unique combination of species, isotype and subclass, and wherein the second binding motif forms a covalent bond with a first binding motif when brought into contact with one another either spontaneously or with the help of an enzyme.

Various embodiments of the first binding motif and the second binding motif discussed above in connection with the full-length antibodies of the invention are also applicable to the nucleic acid constructs of the invention.

The nucleic acid constructs are typically present in various vectors. The vectors of the present invention generally comprise transcriptional or translational control sequences required for expressing the fusion proteins comprising antigen binding fragments and Fc fragments. Suitable transcription or translational control sequences include but are not limited to replication origin, promoter, enhancer, repressor binding regions, transcription initiation sites, ribosome binding sites, translation initiation sites, and termination sites for transcription and translation.

The origin of replication (generally referred to as an ori sequence) permits replication of the vector in a suitable host cell. The choice of ori will depend on the type of host cells and/or genetic packages that are employed. Where the host cells are prokaryotes, the expression vector typically comprises ori sequences directing autonomous replication of the vector within the prokaryotic cells. Preferred prokaryotic ori is capable of directing vector replication in bacterial cells. Non-limiting examples of this class of ori include pMB1, pUC, as well as other E. coli origins.

In the eukaryotic system, higher eukaryotes contain multiple origins of DNA replication, but the ori sequences are not clearly defined. The suitable origins of replication for mammalian vectors are normally from eukaryotic viruses. Preferred eukaryotic ori include, but are not limited to, SV40 ori, EBV ori, or HSV ori. Eukaryotic vectors and eukaryotic host cells are typically used to express Fc fragment-binding motif fusion proteins.

As used herein, a “promoter” is a DNA region capable under certain conditions of binding RNA polymerase and initiating transcription of a coding region located downstream (in the 3′ direction) from the promoter. It can be constitutive or inducible. In general, the promoter sequence is bounded at its 3′ terminus by the transcription initiation site and extends upstream (5′ direction) to include the minimum number of bases or elements necessary to initiate transcription at levels detectable above background. Within the promoter sequence is a transcription initiation site, as well as protein binding domains responsible for the binding of RNA polymerase. Eukaryotic promoters will often, but not always, contain “TATA” boxes and “CAT” boxes.

The choice of promoters will largely depend on the host cells in which the vector is introduced. For prokaryotic cells, a variety of robust promoters are known in the art. Preferred promoters are lac promoter, Trc promoter, T7 promoter and pBAD promoter. Normally, to obtain expression of exogenous sequence in multiple species, the prokaryotic promoter can be placed immediately after the eukaryotic promoter, or inside an intron sequence downstream of the eukaryotic promoter.

Suitable promoter sequences for eukaryotic cells include the promoters for 3-phosphoglycerate kinase, or other glycolytic enzymes, such as enolase, glyceraldehyde-3-phosphate dehydrogenase, hexokinase, pyruvate decarboxylase, phosphofructokinase, glucose-6phosphate isomerase, 3-phosphoglycerate mutase, pyruvate kinase, triosephosphate isomerase, phosphoglucose isomerase, and glucokinase. Other promoters, which have the additional advantage of transcription controlled by growth conditions, are the promoter regions for alcohol dehydrogenase 2, isocytochrome C, acid phosphatase, degradative enzymes associated with nitrogen metabolism, and the aforementioned glyceraldehyde-3-phosphate dehydrogenase, and enzymes responsible for maltose and galactose utilization. Preferred promoters for mammalian cells are SV40 promoter, CMV promoter, β-actin promoter and their hybrids. Preferred promoters for yeast cell includes but is not limited to GAL 10, GAL I, TEFI in S. cerevisiae, and GAP, AOX1 in P. pastoris.

In constructing the subject vectors, the termination sequences associated with the protein coding sequence can also be inserted into the 3′ end of the sequence desired to be transcribed to provide polyadenylation of the mRNA and/or transcriptional termination signal. The terminator sequence preferably contains one or more transcriptional termination sequences (such as polyadenylation sequences) and may also be lengthened by the inclusion of additional DNA sequence so as to further disrupt transcriptional read-through. Preferred terminator sequences (or termination sites) of the present invention have a gene that is followed by a transcription termination sequence, either its own termination sequence or a heterologous termination sequence. Examples of such termination sequences include stop codons coupled to various yeast transcriptional termination sequences or mammalian polyadenylation sequences that are known in the art and are widely available. Where the terminator comprises a gene, it can be advantageous to use a gene which encodes a detectable or selectable marker; thereby providing a means by which the presence and/or absence of the terminator sequence (and therefore the corresponding inactivation and/or activation of the transcription unit) can be detected and/or selected.

In addition to the above-described elements, the vectors may contain a selectable marker (for example, a gene encoding a protein necessary for the survival or growth of a host cell transformed with the vector), although such a marker gene can be carried on another polynucleotide sequence co-introduced into the host cell. Only those host cells into which a selectable gene has been introduced will survive and/or grow under selective conditions. Typical selection genes encode protein(s) that (a) confer resistance to antibiotics or other toxins, e.g., ampicillin, kanamycin, neomycin, zeocin, G418, methotrexate, etc.; (b) complement auxotrophic deficiencies; or (c) supply critical nutrients not available from complex media. The choice of the proper marker gene will depend on the host cell, and appropriate genes for different hosts are known in the art.

In one embodiment, the expression vector is a shuttle vector, capable of replicating in at least two unrelated host systems. In order to facilitate such replication, the vector generally contains at least two origins of replication, one effective in each host system. Typically, shuttle vectors are capable of replicating in a eukaryotic host system and a prokaryotic host system. This enables detection of protein expression in the eukaryotic host (the expression cell type) and amplification of the vector in the prokaryotic host (the amplification cell type). Preferably, one origin of replication is derived from SV40 or 2u and one is derived from pUC, although any suitable origin known in the art may be used provided it directs replication of the vector. Where the vector is a shuttle vector, the vector preferably contains at least two selectable markers, one for the expression cell type and one for the amplification cell type. Any selectable marker known in the art or those described herein may be used provided it functions in the expression system being utilized.

The vectors encompassed by the invention can be obtained using recombinant cloning methods and/or by chemical synthesis. A vast number of recombinant cloning techniques such as PCR, restriction endonuclease digestion and ligation are well known in the art, and need not be described in detail herein. One of skill in the art can also use the sequence data provided herein or that in the public or proprietary databases to obtain a desired vector by any synthetic means available in the art. Additionally, using well-known restriction and ligation techniques, appropriate sequences can be excised from various DNA sources and integrated in operative relationship with the exogenous sequences to be expressed in accordance with the present invention.

### Definitions:

As used in this specification and claims, the singular form “a,” “an,” and “the” include plural references unless the context clearly dictates otherwise.

A “full-length antibody” refers to an antibody-like molecule having at least one antigen binding fragment and at least one Fc fragment.

An antigen binding fragment refers to the antigen binding portion of an antibody, such as a Fab fragment, a variable fragment (Fv), a single-chain variable fragment (scFv), a single-chain Fab fragment (scFab), or any single domain antibody (sdAbs) such as a camelid VHH or a shark Variable New Antigen Receptor (VNAR) domain. It also refers to other proteinaceous affinity reagents or binding scaffolds that are not derived from the antibody immunoglobulin fold such as Variable Lymphocyte Receptors (VLRs), affimers, affibodies, darpins, anticalins, monobodies.

An Fc fragment is defined as the tail region of an antibody that interacts with Fc receptors and some proteins of the complement system, and which can be detected by corresponding Fc-specific secondary antibodies in immunoassays. Fc fragments may or may not contain a hinge sequence at the N-terminus. Fc fragments typically consist of two or more constant domains that form soluble homodimers or higher-order structures of such homodimers.

The term “binding motif” relates to a protein sequence that is attached to an Fc fragment and to an antigen binding fragment and that facilitates the formation of a covalent linkage to conjugate the Fc fragment and the antigen binding fragment to produce a full-length antibody. Non-limiting examples of binding motifs include SpyTag sequences, including SpyTag002 (SEQ ID NO: 34) and SpyTag003 (SEQ ID NO: 43), SpyCatcher sequence, including SpyCatcher002 sequence and SpyCatcher003 sequence, SnoopTag sequences, SnoopCatcher sequence, Sortase motifs, butelase substrates, and peptiligase substrates. The binding motifs may be fused to an Fc fragment at the N-terminus or to an antigen binding fragment at the C-terminus. Alternatively, the binding motifs may be fused to an Fc fragment at the C-terminus and to an antigen binding fragment at the N-terminus, or to an Fc fragment at the C-terminus and to an antigen binding fragment at the C-terminus. A spacer sequence (e.g., a glycine/serine rich spacer) may flank the binding motifs to enhance accessibility for reaction or to enhance flexibility of the antigen binding fragments fused to the Fc.

The term “prokaryotic system” refers to prokaryotic cells such as bacterial cells or prokaryotic viruses, prokaryotic phages or bacterial spores. The term “eukaryotic system” refers to eukaryotic cells including cells of animal, plants, fungi and protists, and eukaryotic viruses such as retrovirus, adenovirus, baculovirus. Prokaryotic and eukaryotic systems may be, collectively, referred to as “expression systems”.

The term “expression cassette” is used here to refer to a functional unit that is built in a vector for the purpose of expressing recombinant antigen binding fragments and Fc fragments. An expression cassette includes a promoter or promoters, a transcription terminator sequence, a ribosome binding site or ribosome binding sites, and the cDNA encoding the fusion proteins. Other genetic components can be added to an expression cassette, depending on the expression system (e.g., enhancers and polyadenylation signals for eukaryotic expression systems).

As used herein the term “vector” refers to a nucleic acid molecule, preferably self-replicating, which transfers an inserted nucleic acid molecule into and/or between host cells. Typically vectors are circular DNA comprising a replication origin, a selection marker, and/or viral package signal, and other regulatory elements. Vector, vector DNA, plasmid DNA, phagemid DNA are interchangeable terms in description of this invention. The term includes vectors that function primarily for insertion of DNA or RNA into a cell, replication vectors that function primarily for the replication of DNA or RNA, and expression vectors that function for transcription and/or translation of the DNA or RNA. Also included are vectors that provide more than one of the above functions.

As used herein the term “expression vector” is a polynucleotide which, when introduced into an appropriate host cell, can be transcribed and translated into a polypeptide(s). The term “expression vector”, refers to vectors that direct the expression of Fc fragments or antigen binding fragments of interest fused in frame with a binding motif.

As used herein the terms “polynucleotides”, “nucleic acids”, and “oligonucleotides” are used interchangeably. They refer to a polymeric form of nucleotides of any length, either deoxyribonucleotides or ribonucleotides, or analogs thereof. The following are non-limiting examples of polynucleotides: coding or non-coding regions of a gene or gene fragment, loci (locus) defined from linkage analysis, exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes, and primers. A polynucleotide may comprise modified nucleotides, such as methylated nucleotides and nucleotide analogs. If present, modifications to the nucleotide structure may be imparted before or after assembly of the nucleotide polymer.

As used herein the term “amino acid” refers to either natural and/or unnatural or synthetic amino acids, including glycine and both the D or L optical isomers, and amino acid analogs and peptidomimetics.

As used herein the terms “polypeptide”, “peptide”, and “protein,” are used interchangeably herein to refer to polymers of amino acids of any length.

As used herein the term “host cell” includes an individual cell or cell culture which can be, or has been, a recipient for the disclosed expression constructs. Host cells include progeny of a single host cell. The progeny may not necessarily be completely identical to the original parent cell due to natural, accidental, or deliberate mutation.

TABLE 1
Exemplary first and second binding motifs

| First binding motif | Second binding motif |
|---|---|
| SEQ ID NO: 7 or a sequence with at | SEQ ID NO: 8 or a sequence |
| least 70% identity to SEQ ID NO: 7 | with at least 50% identity to SEQ |
| | ID NO: 8 |
| SEQ ID NO: 7 or a sequence with at | SEQ ID NO: 9 or a sequence |
| least 70% identity to SEQ ID NO: 7 | with at least 50% identity to SEQ |
| | ID NO: 9 |
| SEQ ID NO: 34 or a sequence with at | SEQ ID NO: 8, 9, 28, or 44 or a |
| least 70% identity to SEQ ID NO: 34 | sequence with at least 50% |
| | identity to SEQ ID NO: 8, 9, 28, |
| | or 44 |
| SEQ ID NO: 29 or a sequence with at | SEQ ID NO: 33 or a sequence |
| least 70% identity to SEQ ID NO: 29 | with at least 70% identity to SEQ |
| | ID NO: 33 |
| SEQ ID NO: 37 or a sequence with at | SEQ ID NO: 38 or a sequence |
| least 70% identity thereto SEQ ID | with at least 70% identity thereto |
| NO: 35 or a sequence at least 70% | SEQ ID NO: 36 or a sequence at |
| identical to SEQ ID NO: 35 | least 70% identical to SEQ ID |
| | NO: 36 |
| SEQ ID NO: 39 or a sequence with at | SEQ ID NO: 40 or a sequence |
| least 70% identity thereto SEQ ID | with at least 50% identity thereto |
| NO: 41 or a sequence with at least | SEQ ID NO: 42 or a sequence |
| 70% identity thereto | with at least 50% identity thereto |
| SEQ ID NO: 43 or a sequence with at | SEQ ID NO: 8, 9, 28, or 44 or a |
| least 70% identity thereto | sequence with at least 50% |
| | identity to SEQ ID NO: 8, 9, 28, |
| | or 44. |
| SEQ ID NO: 25 or 27 or a sequence | SEQ ID NO: 26 or a sequence |
| with at least 50% identity to SEQ ID | with at least 50% identity thereto |
| NO: 25 or 27, that is 15 to 50 amino | or SEQ ID NO: 28 or a sequence |
| acids in length and contain an | with at least 50% identity thereto |
| aspartic acid corresponding to | |
| position 8 in SEQ ID NO: 27 | |
| a fragment of SEQ ID NO: 6 that | fragments of SEQ ID NO: 6 that |
| includes the aspartic acid residue | contain the reactive lysine of |
| corresponding to position 101 (or a | position 15 (or sequences at least |
| sequence at least 70% identical | 50% identical thereto) |
| thereto) | |
| fragments of SEQ ID NO: 5 that | fragments of SEQ ID NO: 5 that |
| include the asparagine corresponding | have at least 50% sequence |
| to position 266 (or sequences having | identity thereto and which |
| at least 50% identity thereto) | comprise the lysine residue at |
| | position 149 but which do not |
| | include the asparagine at position |
| | 266 |
| residues 179-184 or 173-185 of SEQ | residues 191-317 or 186-318 of |
| ID NO: 3 or a sequence with at least | SEQ ID NO: 3 or a sequence |
| 50% identity thereto and which is | having at least 50% identity |
| less than 50 amino acids in length | thereto, excluding the full length |
| | of SEQ ID NO: 3 |
| residues 31-40 of SEQ ID NO: 1 or a | residues 37-304 of SEQ ID NO: |
| sequence with at least 70% identity | 1 or a sequence with at least 70% |
| thereto and less than 50 amino acids | identity thereto, excluding the |
| in length | full length sequence of SEQ ID |
| | NO: 1 |
| residues 302-308 of SEQ ID NO: 1 or | residues 31-291 of SEQ ID NO: |
| SEQ ID NO: 25 or SEQ ID NO: 27, | 1 or SEQ ID NO: 26 or SEQ ID |
| or a sequence with at least 50% | NO: 28 or a sequence with at |
| identity to residues 302-308 of SEQ | least 50% identity thereto, |
| ID NO: 1 or 25 or 27 that are less | excluding the full length of SEQ |
| than 50 amino acids in length and | ID NO: 1 |
| which contains the asparagine residue | |
| corresponding to position 303 (as | |
| defined in SEQ ID NO: 1), residues | |
| 301-308, 300-308, 299-308, 298-308, | |
| 297-308, 296-308, 295-308, 294-308, | |
| 293-308, 292-308, 291-308 or 290- | |
| 308 of SEQ ID NO: 1 or a sequence | |
| with at least about 50% identity thereto | |
| and which contain the asparagine | |
| residue corresponding to position 303 | |
| (as defined in SEQ ID NO: 1). | |
| residues 302-308 of SEQ ID NO: 1 or | residues 31-292, 31-293, 31-294, |
| SEQ ID NO: 25 or SEQ ID NO: 27, | 31-295, 31-296, 31-297, 31-298, |
| or a sequence with at least 50% | 31-299, 31-300, 31-301 or 31- |
| identity to residues 302-308 of SEQ | 302 of SEQ ID NO: 1 or a |
| ID NO: 1 or 25 or 27, that are less | sequence with at least 70% |
| than 50 amino acids in length and | identity thereto, excluding the |
| which contain the asparagine residue | full length of SEQ ID NO: 1 |
| corresponding to position 303 (as | |
| defined in SEQ ID NO: 1), residues | |
| 301-308, 300-308, 299-308, 298-308, | |
| 297-308, 296-308, 295-308, 294-308, | |
| 293-308, 292-308, 291-308 or 290- | |
| 308 of SEQ ID NO: 1 or a sequence | |
| with at least about 50% identity | |
| thereto and which contain the | |
| asparagine residue corresponding to | |
| position 303 (as defined in SEQ ID | |
| NO: 1). | |

### ADDITIONAL DISCLOSURE AND CLAIMABLE SUBJECT MATTER

Item 1. A full-length antibody comprising antigen binding fragments comprising a first binding motif at the C-terminus and an Fc fragment comprising a second binding motif at the N-terminus, wherein the first binding motif and the second binding motif are covalently conjugated to each other via protein ligation, with the proviso that if the antigen binding fragment and the Fc fragment are obtained from the same species, the Fc fragment is labeled with a detectable label.

Item 2. The full-length antibody of item 1, wherein the antigen binding fragment is obtained from a first species and the Fc fragment is obtained from a second species that is different from the first species.

Item 3. A plurality of full-length antibodies, wherein each full-length antibody comprises antigen binding fragments comprising a first binding motif at the C-terminus and an Fc fragment comprising a second binding motif at the N-terminus, wherein the first binding motif and the second binding motif are covalently conjugated to each other via protein ligation.

Item 4. The plurality of full-length antibodies of item 3, wherein each antigen binding fragment specifically binds to a unique antigen and each Fc fragment belongs to a unique combination of species, isotype and subclass.

Item 5. The plurality of full-length antibodies of item 3 or 4, wherein each of the full-length antibodies is conjugated to a unique label.

Item 6. The plurality of full-length antibodies of item 3 or 4, wherein each of the full-length antibodies is conjugated to a unique bead.

Item 7. The plurality of full-length antibodies of any one of items 3 to 6, wherein one of the first binding motif and/or the second binding motif comprises SEQ ID NO: 1 or 3 or 5 or 6 or 7 or 25 or 27 or 29 or 30 or 34 or 35 or 37 or 39 or 41 or 43, residues 302-308 of the sequence set out in SEQ ID NO: 1, or a sequence with at least 50% identity to SEQ ID NO: 1 or 3 or 5 or 6 or 7 or 25 or 27 or 29 or 30 or 34 or 35 or 37 or 39 or 41 or 43; or a fragment thereof, and the other binding motif comprises residues 31-291 of the sequence set out in SEQ ID NO: 1, SEQ ID NO: 8 or 9 or 26 or 28 or 33 or 36 or 38 or 40 or 42 or 44 or a sequence with at least 50% identity to SEQ ID NO: 1 or 8 or 9 or 26 or 28 or 33 or 36 or 38 or 40 or 42 or 44; or a fragment thereof, and wherein the first binding motif and the second binding motif interact with each other via protein ligation, either spontaneously or with the help of an enzyme, to form a covalent bond.

Item 8. The plurality of full-length antibodies of item 7, wherein the fragment of SEQ ID NO: 1 or 3 or 5 or 6 comprises about 5-50 amino acids.

Item 9. The plurality of full-length antibodies of item 7 or 8, wherein one of the first binding motif and the second binding motif comprises residues 302-308, 301-308, 300-308, 299-308, 298-308, 297-308, 296-308, 295-308, 294-308, 293-308, 292-308, 291-308 or 290-308 of SEQ ID NO: 1 or a sequence with at least about 50% to 95% identity to residues 302-308 of SEQ ID NO: 1, and wherein the first binding motif and the second binding motif interact with each other via protein ligation, either spontaneously or with the help of an enzyme, to form a covalent bond.

Item 10. The plurality of full-length antibodies of any one of items 3 to 6, wherein the first binding motif or the second binding motif comprises the reactive asparagine of position 303 of SEQ ID NO: 1, and wherein the first binding motif and the second binding motif interact with each other via protein ligation, either spontaneously or with the help of an enzyme, to form a covalent bond.

Item 11. The plurality of full-length antibodies of any one of items 3 to 6, wherein one of the first binding motif and the second binding motif comprises a fragment of SEQ ID NO: 1 comprising the reactive lysine residue at position 36 of SEQ ID NO: 1 and the other binding motif comprises a fragment of SEQ ID NO: 1 comprising the reactive asparagine at position 168 of SEQ ID NO: 1, and wherein the first binding motif and the second binding motif interact with each other via protein ligation, either spontaneously or with the help of an enzyme, to form a covalent bond.

Item 12. The plurality of full-length antibodies of any one of items 3 to 6, wherein one of the first binding motif and the second binding motif comprises a fragment of SEQ ID NO: 5 comprising the reactive lysine residue at position 149 of SEQ ID NO: 5 and the other binding motif comprises a fragment of SEQ ID NO: 5 comprising the reactive asparagine at position 266 of SEQ ID NO: 5, and wherein the first binding motif and the second binding motif interact with each other via protein ligation, either spontaneously or with the help of an enzyme, to form a covalent bond.

Item 13. The plurality of full-length antibodies of any one of items 3 to 6, wherein one of the first binding motif and the second binding motif comprises a fragment of SEQ ID NO: 6 comprising the reactive lysine residue at position 15 of SEQ ID NO: 6 and the other binding motif comprises a fragment of SEQ ID NO: 6 comprising the reactive aspartic acid at position 101 of SEQ ID NO: 6, and wherein the first binding motif and the second binding motif interact with each other via protein ligation, either spontaneously or with the help of an enzyme, to form a covalent bond.

Item 14. The plurality of full-length antibodies of any one of items 3 to 6, wherein one of the first binding motif and the second binding motif comprises a fragment of SEQ ID NO: 1 comprising the reactive asparagine at position 303 of SEQ ID NO: 1 and the other binding motif comprises a fragment of SEQ ID NO: 1 comprising the reactive lysine at position 179 of SEQ ID NO: 1, and wherein the first binding motif and the second binding motif interact with each other via protein ligation, either spontaneously or with the help of an enzyme, to form a covalent bond.

Item 15. The plurality of full-length antibodies of any one of items 3 to 6, wherein one of the first binding motif and the second binding motif comprises a fragment of SEQ ID NO: 1 comprising the reactive lysine at position 36 of SEQ ID NO: 1 and the other binding motif comprises a fragment of SEQ ID NO: 1 comprising the reactive asparagine at position 168 of SEQ ID NO: 1, and wherein the first binding motif and the second binding motif interact with each other via protein ligation, either spontaneously or with the help of an enzyme, to form a covalent bond.

Item 16. The plurality of full-length antibodies of any one of items 3 to 6, wherein one of the first binding motif and the second binding motif comprises a fragment of SEQ ID NO: 3 comprising the reactive lysine at position 181 of SEQ ID NO: 3 and the other binding motif comprises a fragment of SEQ ID NO: 3 comprising the reactive asparagine at position 294 of SEQ ID NO: 3, and wherein the first binding motif and the second binding motif interact with each other via protein ligation, either spontaneously or with the help of an enzyme, to form a covalent bond.

Item 17. The plurality of full-length antibodies of any one of items 3 to 6, wherein one of the first binding motif and the second binding motif comprises a fragment of SEQ ID NO: 10 comprising the reactive lysine at position 176 of SEQ ID NO: 10 and the other binding motif comprises a fragment of SEQ ID NO: 10 comprising the reactive asparagine at position 308 of SEQ ID NO: 10, and wherein the first binding motif and the second binding motif interact with each other via protein ligation, either spontaneously or with the help of an enzyme, to form a covalent bond.

Item 18. The plurality of full-length antibodies of any one of items 3 to 6, wherein one of the first binding motif and the second binding motif comprises a fragment of SEQ ID NO: 11 comprising the reactive lysine at position 15 of SEQ ID NO: 11 and the other binding motif comprises a fragment of SEQ ID NO: 11 comprising the reactive aspartic acid at position 101 of SEQ ID NO: 11, and wherein the first binding motif and the second binding motif interact with each other via protein ligation, either spontaneously or with the help of an enzyme, to form a covalent bond.

Item 19. The plurality of full-length antibodies of any one of items 3 to 6, wherein one of the first binding motif and the second binding motif comprises a fragment of SEQ ID NO: 13 comprising the reactive lysine at position 742 of SEQ ID NO: 13 and the other binding motif comprises a fragment of SEQ ID NO: 13 comprising the reactive asparagine at position 854 of SEQ ID NO: 13, and wherein the first binding motif and the second binding motif interact with each other via protein ligation, either spontaneously or with the help of an enzyme, to form a covalent bond.

Item 20. The plurality of full-length antibodies of any one of items 3 to 6, wherein one of the first binding motif and the second binding motif comprises a fragment of SEQ ID NO: 15 comprising the reactive lysine at position 405 of SEQ ID NO: 15 and the other binding motif comprises a fragment of SEQ ID NO: 15 comprising the reactive aspartic acid at position 496 of SEQ ID NO: 15.

Item 21. The plurality of full-length antibodies of any one of items 3 to 6, wherein the first binding motif and/or the second binding motif comprises an isopeptide comprising an amino acid sequence of SEQ ID NO: 21 or 23 or 25 or 27 or a protein with at least 70% sequence identity to an amino acid sequence as set forth in any one of SEQ ID NO: 21 or 23 or 25 or 27.

Item 22. The plurality of full-length antibodies of any one of items 3 to 6, wherein the first binding motif comprises a sortase recognition domain and the second binding motif comprises a sortase bridging domain.

Item 23. The plurality of full-length antibodies of item 22, wherein the sortase recognition domain comprises the amino acid sequence: LPTGAA (SEQ ID NO: 18), LPTGGG (SEQ ID NO: 19), LPKTGG (SEQ ID NO: 20), LPETG (SEQ ID NO: 21), LPXTG (SEQ ID NO: 22) or LPXTG (X) n (SEQ ID NO: 23), where X is any amino acid, and n is 0, 1, 2, 3, 4, 5, 7, 8, 9, 10, in the range of 0-5 or 0-10, or any integer up to 100, NPX1TX2 (SEQ ID NO: 24), where X1 is glutamine or lysine; X2 is asparagine or glycine; N is asparagine; P is proline and T is threonine, and the sortase bridging domain comprises: Gly, (Gly)₂, (Gly)₃, (Gly)₄, or (Gly)ₓ, where x is an integer of 1-20.

Item 24. The plurality of full-length antibodies of any one of items 3 to 6, wherein the first binding motif comprises a butelase recognition domain.

Item 25. The plurality of full-length antibodies of item 24, wherein the butelase recognition domain comprises the amino acid sequence: Asn-His-Val or Asp-His-Val.

Item 26. The plurality of full-length antibodies of any one of items 3 to 6, wherein the first binding motif and the second binding motif each comprises a split intein, and wherein the first binding motif and the second binding motif interact with each other via protein ligation, either spontaneously or with the help of an enzyme, to form a covalent bond.

Item 27. A method of determining the levels of a plurality of antigens in a sample, comprising contacting the sample with a plurality of full-length antibodies of any of claims 3 to 26, and quantifying the binding between each of the plurality of full-length antibodies and their corresponding antigens to determine the presence and the levels of the plurality antigens in the sample.

Item 28. A plurality of pairs of nucleic acid constructs, wherein each pair of nucleic acid construct comprises:
a) a first nucleic acid construct comprising a polynucleotide sequence encoding an antigen binding fragment fused at the C-terminus to a first binding motif; and
b) a second nucleic acid construct comprising a polynucleotide encoding an Fc fragment fused at the N-terminus to a second binding motif,
wherein each antigen binding fragment specifically binds to a unique antigen and each Fc fragment belongs to a unique combination of species, isotype and subclass, and
wherein the first binding motif and the second binding motif form a covalent bond when brought into contact with one another either spontaneously or with the help of an enzyme.

Item 29. The combination of pairs of nucleic acid constructs according to item 28, wherein:
a) one of the first binding motif and the second binding motif comprises SEQ ID NO: 1 or 3 or 5 or 6 or 7 or 25 or 27 or 29 or 30 or 34 or 35 or 37 or 39 or 41 or 43, residues 302-308 of the sequence set out in SEQ ID NO: 1, or a sequence with at least 50% identity to SEQ ID NO: 1 or 3 or 5 or 6 or 7 or 25 or 27 or 29 or 30 or 34 or 35 or 37 or 39 or 41 or 43; or a fragment thereof; and the other binding motif comprises residues 31-291 of the sequence set out in SEQ ID NO: 1, SEQ ID NO: 8 or 9 or 26 or 28 or 33 or 36 or 38 or 40 or 42 or 44 or a sequence with at least 50% identity to SEQ ID NO: 1 or 8or 9 or 26 or 28 or 33 or 36 or 38 or 40 or 42 or 44; or a fragment thereof,
b) one of the first binding motif and the second binding motif comprises residues 302-308, 301-308, 300-308, 299-308, 298-308, 297-308, 296-308, 295-308, 294-308, 293-308, 292-308, 291-308 or 290-308 of SEQ ID NO: 1 or a sequence with at least about 50% to 95% identity to residues 302-308 of SEQ ID NO: 1;
c) the first binding motif or the second binding motif comprises the reactive asparagine of position 303 in SEQ ID NO: 1;
d) one of the first binding motif and the second binding motif comprises a fragment of SEQ ID NO: 1 comprising the reactive lysine residue at position 36 of SEQ ID NO: 1 and the other binding motif comprises a fragment of SEQ ID NO: 1 comprising the reactive asparagine at position 168 of SEQ ID NO: 1;
e) one of the first binding motif and the second binding motif comprises a fragment of SEQ ID NO: 5 comprising the reactive lysine residue at position 149 of SEQ ID NO: 5 and the other binding motif comprises a fragment of SEQ ID NO: 5 comprising the reactive asparagine at position 266 of SEQ ID NO: 5;
f) one of the first binding motif and the second binding motif comprises a fragment of SEQ ID NO: 6 comprising the reactive lysine residue at position 15 of SEQ ID NO: 6 and the other binding motif comprises a fragment of SEQ ID NO: 6 comprising the reactive aspartic acid at position 101 of SEQ ID NO: 6;
g) one of the first binding motif and the second binding motif comprises a fragment of SEQ ID NO: 1 comprising the reactive asparagine at position 303 of SEQ ID NO: 1 and the other binding motif comprises a fragment of SEQ ID NO: 1 comprising the reactive lysine at position 179 of SEQ ID NO: 1;
h) one of the first binding motif and the second binding motif comprises a fragment of SEQ ID NO: 1 comprising the reactive lysine at position 36 of SEQ ID NO: 7 and the other binding motif comprises a fragment of SEQ ID NO: 1 comprising the reactive asparagine at position 168 of SEQ ID NO: 1;
i) one of the first binding motif and the second binding motif comprises a fragment of SEQ ID NO: 3 comprising the reactive lysine at position 181 of SEQ ID NO: 3 and the other binding motif comprises a fragment of SEQ ID NO: 3 comprising the reactive asparagine at position 294 of SEQ ID NO: 3;
j) one of the first binding motif and the second binding motif comprises a fragment of SEQ ID NO: 10 comprising the reactive lysine at position 176 of SEQ ID NO: 10 and the other binding motif comprises a fragment of SEQ ID NO: 10 comprising the reactive asparagine at position 308 of SEQ ID NO: 10;
k) one of the first binding motif and the second binding motif comprises a fragment of SEQ ID NO: 11 comprising the reactive lysine at position 15 of SEQ ID NO: 11 and the other binding motif comprises a fragment of SEQ ID NO: 11 comprising the reactive aspartic acid at position 101 of SEQ ID NO: 11;
l) one of the first binding motif and the second binding motif comprises a fragment of SEQ ID NO: 13 comprising the reactive lysine at position 742 of SEQ ID NO: 13 and the other binding motif comprises a fragment of SEQ ID NO: 13 comprising the reactive asparagine at position 854 of SEQ ID NO: 13;
m) one of the first binding motif and the second binding motif comprises a fragment of SEQ ID NO: 15 comprising the reactive lysine at position 405 of SEQ ID NO: 15 and the other binding motif comprises a fragment of SEQ ID NO: 15 comprising the reactive aspartic acid at position 496 of SEQ ID NO: 15;
n) the first binding motif and/or the second binding motif comprises an isopeptide comprising an amino acid sequence of SEQ ID NO: 21 or 23 or 25 or 27 or a protein with at least 70% sequence identity to an amino acid sequence as set forth in any one of SEQ ID NO: 21 or 23 or 25 or 27;
o) the first binding motif comprises a sortase recognition domain and the second binding motif comprises a sortase bridging domain;
p) the first binding motif comprises a Butelase 1 recognition domain; or
q) the first binding motif and the second binding motif each comprises a split intein;
and wherein the first binding motif and the second binding motif interact with each other via protein ligation, either spontaneously or with the help of an enzyme, to form a covalent bond.

Item 30. A plurality of prokaryotic or eukaryotic host cells, wherein each of the plurality of prokaryotic or eukaryotic host cells comprises one nucleic acid constructs from the nucleic acid constructs according to claim 28 or 29.

Item 31. A plurality of Fc fragments, wherein each Fc fragment comprises a unique second binding motif at the N-terminus, wherein each unique second binding motif is capable covalently conjugating via protein ligation, either spontaneously or with the help of an enzyme, to a unique first binding motif, and wherein each Fc fragment belongs to a unique combination of species, isotype and/or subclass.

Item 32. The plurality Fc fragments of item 31, wherein each of the Fc fragments is conjugated to a unique label.

Item 33. The plurality Fc fragments of item 31, wherein each of the Fc fragments is conjugated to a unique bead.

Item 34. The plurality of Fc fragments of any one of items 31 to 33, wherein:
i) the unique second binding motif comprises SEQ ID NO: 1 or 3 or 5 or 6 or 7 or 25 or 27 or 29 or 30 or 34 or 35 or 37 or 39 or 41 or 43, residues 302-308 of the sequence set out in SEQ ID NO: 1, or a sequence with at least 50% identity to SEQ ID NO: 1 or 3 or 5 or 6 or 7 or 25 or 27 or 29 or 30 or 34 or 35 or 37 or 39 or 41 or 43; or a fragment thereof, and is capable of is capable covalently conjugating via protein ligation, either spontaneously or with the help of an enzyme, to a unique first binding motif comprising residues 31-291 of the sequence set out in SEQ ID NO: 1 or 8 or 9 or 26 or 28 or 33 or 36 or 38 or 40 or 42 or 44 or a sequence with at least 50% identity to SEQ ID NO: 1 or 8 or 9 or 26 or 28 or 33 or 36 or 38 or 40 or 42 or 44; or a fragment thereof, or
ii) the unique second binding motif comprises residues 31-291 of the sequence set out in SEQ ID NO: 1 or 8 or 9 or 28 or 33 or 36 or 38 or 40 or 42 or 44 or a sequence with at least 50% identity to SEQ ID NO: 1 or 8 or 9 or 26 or 28 or 33 or 36 or 38 or 40 or 42 or 44; or a fragment thereof; and is capable covalently conjugating via protein ligation, either spontaneously or with the help of an enzyme, to a unique first binding motif comprising SEQ ID NO: 1 or 3 or 5 or 6 or 7 or 25 or 27 or 29 or 30 or 34 or 35 or 37 or 39 or 41 or 43, residues 302-308 of the sequence set out in SEQ ID NO: 1, or a sequence with at least 50% identity to SEQ ID NO: 1 or 3 or 5 or 6 or 7 or 25 or 27 or 29 or 30 or 34 or 35 or 37 or 39 or 41 or 43; or a fragment thereof.

Item 35. The plurality of Fc fragments of item 34, wherein the fragment of SEQ ID NO: 1 or 3 or 5 or 6 comprises about 5-50 amino acids.

Item 36. The plurality of Fc fragments of item 34 or 35, wherein the unique second binding motif comprises residues 302-308, 301-308, 300-308, 299-308, 298-308, 297-308, 296-308, 295-308, 294-308, 293-308, 292-308, 291-308 or 290-308 of SEQ ID NO: 1 or a sequence with at least about 50% to 95% identity to residues 302-308 of SEQ ID NO: 1, and wherein the unique second binding motif is capable covalently conjugating via protein ligation, either spontaneously or with the help of an enzyme, to the unique first binding motif.

Item 37. The plurality of Fc fragments of any one of items 31 to 33, wherein the unique second binding motif comprises the reactive asparagine of position 303 of SEQ ID NO: 1 and is capable covalently conjugating via protein ligation, either spontaneously or with the help of an enzyme, to the unique first binding motif.

Item 38. The plurality of Fc fragments of any one of items 31 to 33, wherein:
i) the unique second binding motif comprises a fragment of SEQ ID NO: 1 comprising the reactive lysine residue at position 36 of SEQ ID NO: 1 and is capable covalently conjugating via protein ligation, either spontaneously or with the help of an enzyme, to a unique first binding motif comprising a fragment of SEQ ID NO: 1 comprising the reactive asparagine at position 168 of SEQ ID NO: 1, or
ii) the unique second binding motif comprises a fragment of SEQ ID NO: 1 comprising the reactive the reactive asparagine at position 168 of SEQ ID NO: 1 and is capable covalently conjugating via protein ligation, either spontaneously or with the help of an enzyme, to a unique first binding motif comprising a fragment of SEQ ID NO: 1 comprising the reactive lysine residue at position 36 of SEQ ID NO: 1.

Item 39. The plurality of Fc fragments of any one of items 31 to 33, wherein:
i) the unique second binding motif comprises a fragment of SEQ ID NO: 5 comprising the reactive lysine residue at position 149 of SEQ ID NO: 5 and is capable covalently conjugating via protein ligation, either spontaneously or with the help of an enzyme, to a unique first binding motif comprising a fragment of SEQ ID NO: 5 comprising the reactive asparagine at position 266 of SEQ ID NO: 5, or
ii) the unique second binding motif comprises a fragment of SEQ ID NO: 5 comprising the reactive asparagine at position 266 of SEQ ID NO: 5 and is capable covalently conjugating via protein ligation, either spontaneously or with the help of an enzyme, to a unique first binding motif comprising a fragment of SEQ ID NO: 5 comprising the reactive lysine residue at position 149 of SEQ ID NO: 5.

Item 40. The plurality of Fc fragments of any one of items 31 to 33, wherein:
i) the unique second binding motif comprises a fragment of SEQ ID NO: 6 comprising the reactive lysine residue at position 15 of SEQ ID NO: 6 and is capable covalently conjugating via protein ligation, either spontaneously or with the help of an enzyme, to a unique first binding motif comprising a fragment of SEQ ID NO: 6 comprising the reactive aspartic acid at position 101 of SEQ ID NO: 6, or
ii) the unique second binding motif comprises a fragment of SEQ ID NO: 6 comprising the reactive aspartic acid at position 101 of SEQ ID NO: 6 and is capable covalently conjugating via protein ligation, either spontaneously or with the help of an enzyme, to a unique first binding motif comprising a fragment of SEQ ID NO: 6 comprising the reactive lysine residue at position 15 of SEQ ID NO: 6.

Item 41. The plurality of Fc fragments of any one of items 31 to 33, wherein:
i) the unique second binding motif comprises a fragment of SEQ ID NO: 1 comprising the reactive asparagine at position 303 of SEQ ID NO: 1 and is capable covalently conjugating via protein ligation, either spontaneously or with the help of an enzyme, to a unique first binding motif comprising a fragment of SEQ ID NO: 1 comprising the reactive lysine at position 179 of SEQ ID NO: 1, or
ii) the unique second binding motif comprises a fragment of SEQ ID NO: 1 comprising the reactive lysine at position 179 of SEQ ID NO: 1 and is capable covalently conjugating via protein ligation, either spontaneously or with the help of an enzyme, to a unique first binding motif comprising a fragment of SEQ ID NO: 1 comprising the reactive asparagine at position 303 of SEQ ID NO: 1.

Item 42. The plurality of Fc fragments of any one of items 31 to 33, wherein:
i) the unique second binding motif comprises a fragment of SEQ ID NO: 1 comprising the reactive lysine at position 36 of SEQ ID NO: 1 and is capable covalently conjugating via protein ligation, either spontaneously or with the help of an enzyme, to a unique first binding motif comprising a fragment of SEQ ID NO: 1 comprising the reactive asparagine at position 168 of SEQ ID NO: 1, or
ii) the unique second binding motif comprises a fragment of SEQ ID NO: 1 comprising the reactive asparagine at position 168 of SEQ ID NO: 1 and is capable covalently conjugating via protein ligation, either spontaneously or with the help of an enzyme, to a unique first binding motif comprising a fragment of SEQ ID NO: 1 comprising the reactive lysine at position 36 of SEQ ID NO: 1.

Item 43. The plurality of Fc fragments of any one of items 31 to 33, wherein:
i) the unique second binding motif comprises a fragment of SEQ ID NO: 3 comprising the reactive lysine at position 181 of SEQ ID NO: 3 and is capable covalently conjugating via protein ligation, either spontaneously or with the help of an enzyme, to a unique first binding motif comprising a fragment of SEQ ID NO: 3 comprising the reactive asparagine at position 294 of SEQ ID NO: 3, or
ii) the unique second binding motif comprises a fragment of SEQ ID NO: 3 comprising the reactive asparagine at position 294 of SEQ ID NO: 3 and is capable covalently conjugating via protein ligation, either spontaneously or with the help of an enzyme, to a unique first binding motif comprising a fragment of SEQ ID NO: 3 comprising the reactive lysine at position 181 of SEQ ID NO: 3.

Item 44. The plurality of Fc fragments of any one of items 31 to 33, wherein:
i) the unique second binding motif comprises a fragment of SEQ ID NO: 10 comprising the reactive lysine at position 176 of SEQ ID NO: 10 and is capable covalently conjugating via protein ligation, either spontaneously or with the help of an enzyme, to a unique first binding motif comprising a fragment of SEQ ID NO: 10 comprising the reactive asparagine at position 308 of SEQ ID NO: 10, or
ii) the unique second binding motif comprises a fragment of SEQ ID NO: 10 comprising the reactive asparagine at position 308 of SEQ ID NO: 10 and is capable covalently conjugating via protein ligation, either spontaneously or with the help of an enzyme, to a unique first binding motif comprising a fragment of SEQ ID NO: 10 comprising the reactive lysine at position 176 of SEQ ID NO: 10.

Item 45. The plurality of Fc fragments of any one of items 31 to 33, wherein:
i) the unique second binding motif comprises a fragment of SEQ ID NO: 11 comprising the reactive lysine at position 15 of SEQ ID NO: 11 and is capable covalently conjugating via protein ligation, either spontaneously or with the help of an enzyme, to a unique first binding motif comprising a fragment of SEQ ID NO: 11 comprising the reactive aspartic acid at position 101 of SEQ ID NO: 11, or
ii) the unique second binding motif comprises a fragment of SEQ ID NO: 11 comprising the reactive aspartic acid at position 101 of SEQ ID NO: 11 and is capable covalently conjugating via protein ligation, either spontaneously or with the help of an enzyme, to a unique first binding motif comprising a fragment of SEQ ID NO: 11 comprising the reactive lysine at position 15 of SEQ ID NO: 11.

Item 46. The plurality of Fc fragments of any one of items 31 to 33, wherein:
i) the unique second binding motif comprises a fragment of SEQ ID NO: 13 comprising the reactive lysine at position 742 of SEQ ID NO: 13 and is capable covalently conjugating via protein ligation, either spontaneously or with the help of an enzyme, to a unique first binding motif comprising a fragment of SEQ ID NO: 13 comprising the reactive asparagine at position 854 of SEQ ID NO: 13, or
ii) the unique second binding motif comprises a fragment of SEQ ID NO: 13 comprising the reactive asparagine at position 854 of SEQ ID NO: 13 and is capable covalently conjugating via protein ligation, either spontaneously or with the help of an enzyme, to a unique first binding motif comprising a fragment of SEQ ID NO: 13 comprising the reactive lysine at position 742 of SEQ ID NO: 13.

Item 47. The plurality of Fc fragments of any one of items 31 to 33, wherein:
i) the unique second binding motif comprises a fragment of SEQ ID NO: 15 comprising the reactive lysine at position 405 of SEQ ID NO: 15 and is capable covalently conjugating via protein ligation, either spontaneously or with the help of an enzyme, to a unique first binding motif comprising a fragment of SEQ ID NO: 15 comprising the reactive aspartic acid at position 496 of SEQ ID NO: 15, or
ii) the unique second binding motif comprises a fragment of SEQ ID NO: 15 comprising the reactive aspartic acid at position 496 of SEQ ID NO: 15 and is capable covalently conjugating via protein ligation, either spontaneously or with the help of an enzyme, to a unique first binding motif comprising a fragment of SEQ ID NO: 15 comprising the reactive lysine at position 405 of SEQ ID NO: 15.

Item 48. The plurality of Fc fragments of any one of items 31 to 33, wherein the unique second binding motif comprises an isopeptide comprising an amino acid sequence of SEQ ID NO: 21 or 23 or 25 or 27 or a protein with at least 70% sequence identity to an amino acid sequence as set forth in any one of SEQ ID NO: 21 or 23 or 25 or 27.

Item 49. The plurality of Fc fragments of any one of items 31 to 33, wherein the unique second binding motif comprises a sortase bridging domain and is capable covalently conjugating via protein ligation, either spontaneously or with the help of an enzyme, to a unique first binding motif comprising a sortase recognition domain.

Item 50. The plurality of Fc fragments of item 49, wherein the sortase recognition domain comprises the amino acid sequence: LPTGAA (SEQ ID NO: 18), LPTGGG (SEQ ID NO: 19), LPKTGG (SEQ ID NO: 20), LPETG (SEQ ID NO: 21), LPXTG (SEQ ID NO: 22) or LPXTG(X)n (SEQ ID NO: 23), where X is any amino acid, and n is 0, 1, 2, 3, 4, 5, 7, 8, 9, 10, in the range of 0-5 or 0-10, or any integer up to 100, NPX1TX2 (SEQ ID NO: 24), where X1 is glutamine or lysine; X2 is asparagine or glycine; N is asparagine; P is proline and T is threonine, and the sortase bridging domain comprises: Gly, (Gly)2, (Gly)3, (Gly)4, or (Gly)x, where x is an integer of 1-20.

Item 51. The plurality of Fc fragments of any one of items 31 to 33, wherein the unique second binding motif comprises a first split intein and is capable covalently conjugating via protein ligation, either spontaneously or with the help of an enzyme, to the unique first binding motif comprising a second split intein.

Item 52. A method of preparing a plurality of full-length antibodies, wherein each full-length antibody comprises antigen binding fragments comprising a unique first binding motif at the C-terminus and an Fc fragment comprising a unique second binding motif at the N-terminus, the method comprising contacting a plurality of Fc fragments of any one of items 31 to 51 with a plurality of antigen binding fragments, each antigen binding fragment comprising a unique first binding motif at the C-terminus, the contacting performed under conditions that allow the unique second binding motifs to covalently conjugate via protein ligation, either spontaneously or with the help of an enzyme, to the unique first binding motifs.

Item 53. A kit comprising:
a) an antigen binding fragment containing a first binding motif at its C-terminus, optionally comprising a first detectable label; and
b) an Fc fragment comprising a second binding motif at the N-terminus, optionally comprising a second detectable label; and/or
c) a nucleic acid construct comprising a polynucleotide sequence encoding an antigen binding fragment and/or an Fc fragment as defined in item 53a) and/or 53b),
wherein the first binding motif and the second binding motif are capable of covalent conjugation to each other via protein ligation.

Item 54. The kit of item 53, wherein the first and the second detectable label is, independent from each other, a fluorophore, a fluorescent protein, or an enzyme.

Item 55. A method of producing a full length antibody, the method comprising mixing under appropriate conditions:
a) an antigen binding fragment containing a first binding motif at its C-terminus, and
b) an Fc fragment comprising a second binding motif at the N-terminus, wherein the first binding motif and the second binding motif upon said mixing covalently conjugate with each other via protein ligation.

Item 56. The method of item 55, wherein the antigen binding fragment and/or the Fc fragment comprises a detectable label.

Item 57. The method of item 56, wherein the detectable label is a fluorophore, a fluorescent protein, or an enzyme.

Item 58. The plurality of full-length antibodies of item 7, wherein:
a) the first binding motif comprises SEQ ID NO: 7 or a sequence with at least 70% identity to SEQ ID NO: 7, and the second binding motif comprises SEQ ID NO: 8, 9, 28, 33, or 44 or a sequence with at least 50% identity to SEQ ID NO: 8, 9, 28, 33, or 44;
b) the first binding motif comprises SEQ ID NO: 34 or a sequence with at least 70% identity to SEQ ID NO: 34, and the second binding motif comprises SEQ ID NO: 8, 9, 28, or 44 or a sequence with at least 50% identity to SEQ ID NO: 8, 9, 28, or 44;
c) the first binding motif comprises SEQ ID NO: 35 or a sequence with at least 70% identity to SEQ ID NO: 35, and the second binding motif comprises SEQ ID NO: 36 or a sequence with at least 70% identity to SEQ ID NO: 36;
d) the first binding motif comprises SEQ ID NO: 37 or a sequence with at least 70% identity to SEQ ID NO: 37, and the second binding motif comprises SEQ ID NO: 38 or a sequence with at least 70% identity to SEQ ID NO: 38;
e) the first binding motif comprises SEQ ID NO: 39 or a sequence with at least 70% identity to SEQ ID NO: 39, and the second binding motif comprises SEQ ID NO: 40 or a sequence with at least 50% identity to SEQ ID NO: 40;
f) the first binding motif comprises SEQ ID NO: 41 or a sequence with at least 70% identity to SEQ ID NO: 41, and the second binding motif comprises SEQ ID NO: 42 or a sequence with at least 50% identity to SEQ ID NO: 42; or
g) the first binding motif comprises SEQ ID NO: 43 or a sequence with at least 70% identity to SEQ ID NO: 43, and the second binding motif comprises SEQ ID NO: 8, 9, 28, or 44 or a sequence with at least 50% identity to SEQ ID NO: 8, 9, 28, or 44.

Item 59. The combination of pairs of nucleic acid constructs according to item 29, wherein:
a) one of the first binding motif and the second binding motif comprises SEQ ID NO: 7 or a sequence with at least 70% identity to SEQ ID NO: 7, and the other binding motif comprises SEQ ID NO: 8, 9, 28, 33, or 44 or a sequence with at least 50% identity to SEQ ID NO: 8, 9, 28, 33, or 44;
b) one of the first binding motif and the second binding motif comprises SEQ ID NO: 34 or a sequence with at least 70% identity to SEQ ID NO: 34, and the other binding motif comprises SEQ ID NO: 8, 9, 28, or 44 or a sequence with at least 50% identity to SEQ ID NO: 8, 9, 28, or 44;
c) one of the first binding motif and the second binding motif comprises SEQ ID NO: 35 or a sequence with at least 70% identity to SEQ ID NO: 35, and the other binding motif comprises SEQ ID NO: 36 or a sequence with at least 70% identity to SEQ ID NO: 36;
d) one of the first binding motif and the second binding motif comprises SEQ ID NO: 37 or a sequence with at least 70% identity to SEQ ID NO: 37, and the other binding motif comprises SEQ ID NO: 38 or a sequence with at least 70% identity to SEQ ID NO: 38;
e) one of the first binding motif and the second binding motif comprises SEQ ID NO: 39 or a sequence with at least 70% identity to SEQ ID NO: 39, and the other binding motif comprises SEQ ID NO: 40 or a sequence with at least 50% identity to SEQ ID NO: 40;
f) one of the first binding motif and the second binding motif comprises SEQ ID NO: 41 or a sequence with at least 70% identity to SEQ ID NO: 41, and the other binding motif comprises SEQ ID NO: 42 or a sequence with at least 50% identity to SEQ ID NO: 42; or
g) one of the first binding motif and the second binding motif comprises SEQ ID NO: 43 or a sequence with at least 70% identity to SEQ ID NO: 43, and the other binding motif comprises SEQ ID NO: 8, 9, 28, or 44 or a sequence with at least 50% identity to SEQ ID NO: 8, 9, 28, or 44.

Item 60. The plurality of Fc fragments of item 34, wherein the second binding motif comprises SEQ ID NO: 44 or a sequence with at least 50% identity to SEQ ID NO: 44; and is capable of covalently conjugating via protein ligation, either spontaneously or with the help of an enzyme, to a first binding motif comprising SEQ ID NO: 34 or a sequence with at least 50% identity to SEQ ID NO: 34.

### EXAMPLES

The following examples are provided by way of illustration only and not by way of limitation. Those of skill in the art will readily recognize a variety of non-critical parameters that could be changed or modified to yield essentially the same or similar results.

### Example 1—FcCatcher Construction, Expression, and Purification

FcCatchers were constructed with a SpyCatcher002 (SEQ ID NO: 34) or a SpyCatcher003 (SEQ ID NO: 44) and a human IgG1 (hlgG1) Fc domain, genetically fused via a GSSGS-linker plus the last 5 amino acids from the hlgG1 hinge region, EPKSS. The last cysteine of the hinge region was replaced by a serine. The resulting products are also referred to as hFcCatcher2 (using the SpyCatcher002) and hFcCatcher3 (using the SpyCatcher003). A sequence encoding a signal peptide for secretion into the medium was cloned in front of the

SpyCatcher-Fc sequences. These constructs were cloned into the pMAX vector. The resulting plasmids were transfected into the eukaryotic cell line HKB11 (Cho et al. 2002). Upon three to four hours of incubation at standard conditions, the transfected cultures were fed by adding Bio-Rad's standard feed medium in a 1:1 ratio. On day 6 post-transfection approximately 200 ml culture volume containing the FcCatchers were harvested by centrifugation to remove cell debris and subsequently sterile filtrated. Cleared culture supernatants were submitted to one-step affinity chromatography using a FPLC device. The eluted fractions were neutralized, collected, rebuffered to 1× PBS at pH 7.4 and sterile filtered. The concentrations were determined by UV280 nm measurement using a Nanodrop 2000 device and a molar extinction coefficient calculated from the construct sequences.

Similarly, a mouse IgG2a-FcCatcher (mFcCatcher) and mouse IgG2a-FcCatcher3 (based on SpyCatcher003, mFcCatcher3) as well as a rabbit IgG FcCatcher (rbFcCatcher) and a rabbit IgG-FcCatcher3 were cloned by fusion of the SpyCatcher (SEQ ID NO: 8) or SpyCatcher003(SEQ ID NO: 44) to the respective Fc domain. These constructs were transfected, expressed and purified as described above.

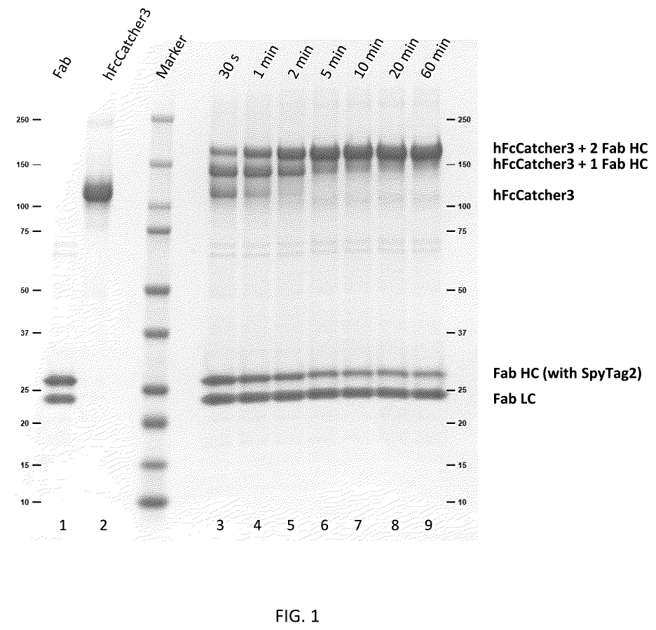

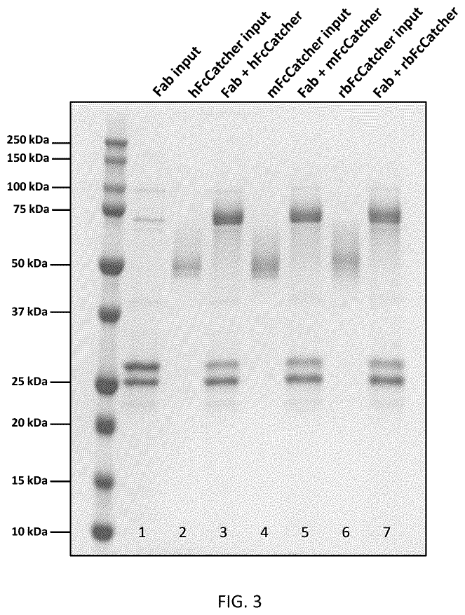

The fusion proteins were then analyzed by SDS-PAGE (FIG. 1 lane 2 and FIG. 3 lanes 2, 4, and 6). A Bio-Rad Criterion™ Vertical Electrophoresis Cell was used along with a 4-20% polyacrylamide gel (Bio-Rad Mini-PROTEAN TGX) and the Bio-Rad Precision Plus Protein Standard molecular weight marker. The gel was stained with Coomassie® stain and the protein purity was determined by densitometry. The concentration and purity of the FcCatchers is provided in TABLE 2 below.

TABLE 2

| Fusion Protein | Yield mg/L | % Purity |
|---|---|---|
| hIgG1-FcSpyCatcher3 | 130 | >90 |
| mIgG2a-FcSpyCatcher3 | 23 | >90 |
| rbIgG-FcSpyCatcher3 | 51 | >90 |

### Example 2—Fab-SpyTag Construction, Expression, and Purification

Human Fab fragments with a FLAG®-tag, SpyTag or SpyTag002 and His-tag were constructed by using a short linker (sequence EF) between the C-terminus of CH1 and FLAG-tag followed by a linker (sequence GGS) and SpyTag or SpyTag002 as well as linker (sequence GAP) and His-tag. Light and heavy chains were cloned into a bicistronic bacterial expression vector with a lac promoter. Both light and heavy chain genes contained secretion signals for transport into the periplasm. Vectors with Fab-FLAG-SpyTag-H or Fab-FLAG-SpyTag2-H constructs were transformed into a protease deficient E. coli strain as described in co-pending U.S. application 62/819,748 (Periplasmic Fusion Proteins; filed Mar. 18, 2019; Docket No. BRL.130P). The Fab fragments were expressed by culturing the E. coli cells in 250 mL 2xYT broth with 0.1% glucose and chloramphenicol. The cultures were induced with 0.8 mM IPTG after 1 hour of growth at 37° C. Expression was allowed to proceed for approximately 16 hours at 30° C. The cultures were centrifuged and the cells were frozen at −80° C. The cells were lysed with BugBuster lysis buffer (Millipore-Sigma). The fusion proteins were purified with Ni-NTA affinity matrix and buffer exchanged into PBS.

### Example 3—Ligation of Fab-SpyTag and FcCatchers

The FcCatcher fusion proteins from Example 1 and the Fab-FLAG-SpyTag2-His fusion proteins from Example 2 were ligated to each other by reacting 10 μM Fab-FLAG-SpyTag2-His with 4 μM of each FcCatcher3 in 1xPBS. A 25% molar excess of SpyTag2 over FcCatcher3 sites (i.e. 2 sites per FcCatcher) was used to achieve complete reaction of all SpyCatcher3 sites. After different time points (between 30 seconds and 60 minutes) the reaction was stopped by adding SDS loading buffer. After heating for 5 minutes at 95° C., samples were loaded onto a 4-20% polyacrylamide gel (Bio-Rad Mini-PROTEAN TGX). An image of the Coomassie stained gel (FIG. 1) shows that the FcCatcher3 reacted with the SpyTag2 at the Fab heavy chain. After 60 minutes the FcCatcher3 band disappeared completely, indicating completion of the ligation reaction. In the beginning of the reaction two products were visible: FcCatcher3 coupled to one

Fab and coupled to two Fabs. The band for the single coupled product diminished with longer reaction times until almost only the double ligated product was visible on the gel after 60 minutes.

### Example 4—Comparison of Assay Performance of Fab-SpyTag2-FcCatcher3 and IgG

Similar performance of Fab-FcCatcher ligation products and IgG was shown by a titration ELISA. A Maxisorp ELISA plate was coated with GFP at lug/ml in PBS overnight. After washing with PBST and blocking with 5% BSA in PBST a titration of an anti-GFP Fab-SpyTag2 ligated to hlgG1-FcSpyCatcher3 in PBST was added to the plate. For comparison, the same antibody (identical Fab sequence) produced in full-length human IgG1 format was titrated at equimolar concentrations. Detection was performed using HRP-conjugated anti-human Fc

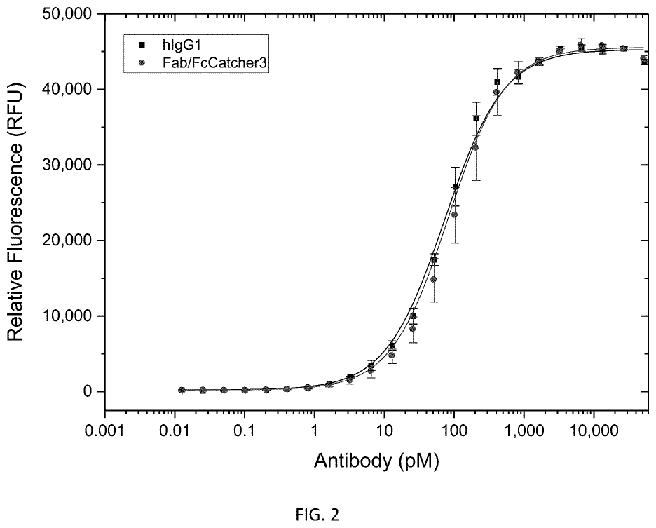

(Bio-Rad MCA647P) at a 1:500 dilution in HiSPEC assay diluent and QuantaBlu fluorogenic peroxidase substrate. The results show that both antibody constructs lead to identical assay sensitivity (FIG. 2).

### Example 5—Immunofluorescence Multiplexing Assay

An immunofluorescence staining of U2OS cells with three human Fabs directed against three different targets (cyclophilin A, vimentin and Ki-67) was performed. All three Fabs were produced in the Fab-FLAG-SpyTag-His format as described in Example 2 and ligated each to hIgG1-FcCatcher, mIgG2a-FcCatcher and rbIgG-FcCatcher from Example 1 with a two-fold molar excess of Fab over FcCatcher overnight. Complete ligation was confirmed by loading the reduced products on an AnykD polyacrylamide gel (Bio-Rad Mini-PROTEAN TGX) together with the Bio-Rad Precision Plus Protein Standard molecular weight marker. FcCatchers reacted completely with the SpyTag at the Fab heavy chain as shown in an image of a Coomassie stained gel of the ligation products (FIG. 3).

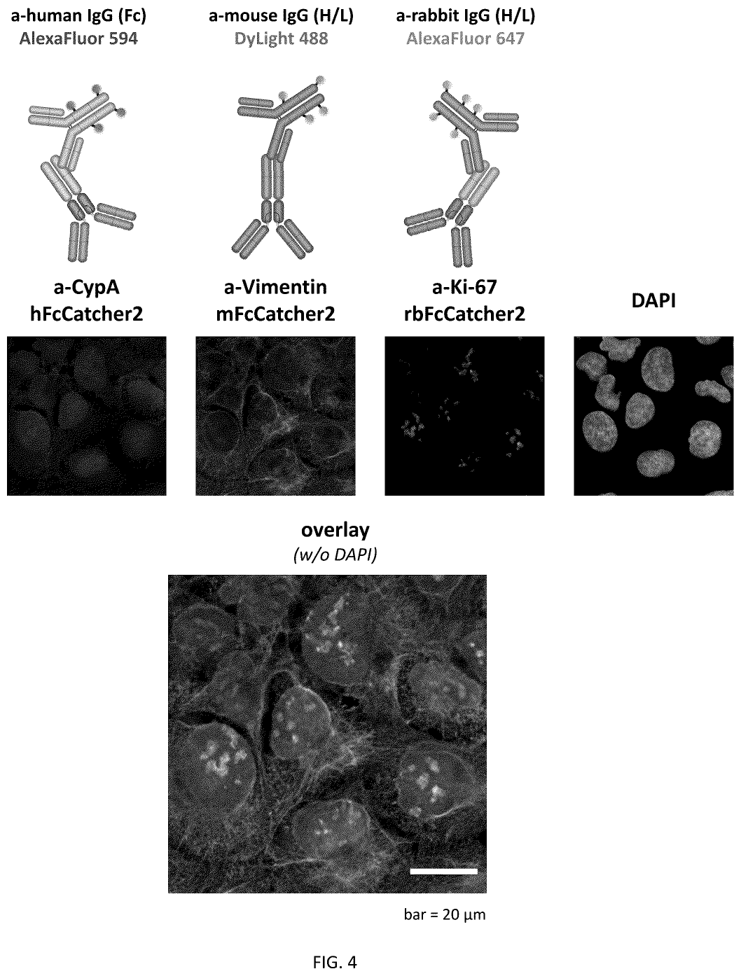

For cell staining, 3.75×10⁴ U2OS cells/well were seeded in 12-well chamber slides with removable silicone gasket (Ibidi). Next day, the cells were fixed with 4% paraformaldehyde in PBS and treated with ice-cold methanol and, subsequently, with 0.2% Triton X-100 and blocked with 5% BSA in PBST for 48 hours at 4° C. The three Fab-FcCatcher ligation products, one of each FcCatcher species, were mixed and added to the cells at 33 nM for anti-Ki-67 and anti-vimentin and 167 nM for anti-cyclophilin A in blocking solution and incubated at room temperature for 3 h. After washing with PBST, blocking solution containing a mixture of three anti-IgG secondary antibodies, consisting of goat anti-hlgG Fc:Alexa Fluor 594 F(ab′)2 (Jackson ImmunoResearch), goat anti-mlgG (H+L):DyLight488 (Bio-Rad), and donkey anti-rbIgG (H+L):Alexa Fluor 647 (Jackson ImmunoResearch) as well as DAPI, was added to the cells and incubated for one hour at room temperature. After washing, cells were mounted in ProLong Gold antifade reagent (Thermo Fisher), cured overnight at room temperature and stored at 4° C. All three possible combinations of the three antibodies and three species were tested. Cells were imaged with a confocal microscope (Zeiss LSM 880 with a EC Plan-Neofluar 40×/1.30 immersion lens) and analysed with Image J software. One set of images is shown in FIG. 4.

### Example 6—Flow Cytometry Multiplexing Assay

Jurkat cells were stained with anti-CD3 and anti-CD45 antibodies. The antibodies were derived from mouse hybridomas and expressed recombinant as Fab with Flag, SpyTag002 and His-tag as described in Example 2. Both antibodies were ligated to human and rabbit FcCatcher3. An excess of 25% Fab was used for the Fab-FcCatcher ligation and incubation time was 1 h.

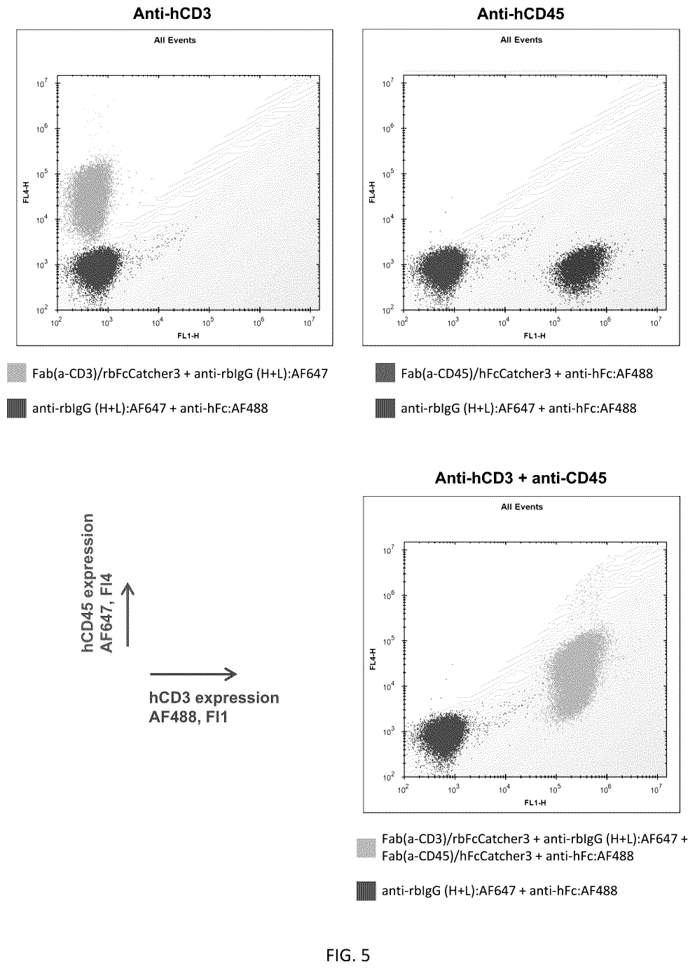

For the assay, 3×10⁴ Jurkat cells in 20 μl flow buffer (3% fetal calf serum (FCS) in PBS) were given into a V-bottom 384 well plate. Fab-FcCatcher3 ligation products were added to the cells at 200 nM final concentration for anti-CD3 and 100 nM for anti-CD45 in a final volume of 60 μl. After incubation for one hour, the cells were washed with flow buffer and a mixture of an Alexa Fluor 488 conjugated anti-human Fc secondary antibody (Jackson ImmunoResearch) and Alexa Fluor 647 conjugated anti-rabbit IgG (H+L) secondary antibody (Jackson ImmunoResearch) was added for one hour at room temperature. Cells were washed and measured on a flow cytometer (IntelliCyt). Analysis of the data showed specific staining for CD3 and CD45 with both antibodies in parallel but no staining with just the two secondary antibodies (FIG. 5).

### REFERENCES

U.S. Pat. No. 9,547,003
WO 2017/058114
WO 2013/045632
Abe, H., Rie, W., Yonemura, H., Yamada, S., Goto, M., and Kamiya, N., (2013), Split Spy0128 as a Potent Scaffold for Protein Cross-Linking and Immobilization. Bioconjugate Chem., 24(2): 242-250.
Alam et al., 2017, Synthetic Modular Antibody Construction Using the SpyTag/SpyCatcher Protein Ligase System. Chembiochem. 18(22), 2217-2221.
Albrecht, H., Burke, P. A., Natarajan, A., Xiong, C. Y., Kalicinsky, M., DeNardo, G. L., et al., 2004, Production of soluble scFvs with C-terminal-free thiol for site-specific conjugation or stable dimeric scFvs on demand. BioconjugChem. 15(1): 16-26.
Batonick, M.,Kiss, M. M., Fuller, E. P., Magadan, C. M., Holland, E. G., Zhao, Q., Wang, D., Kay, B. K., Weiner, M. P., 2016, pMINERVA: A donor-acceptor system for the in vivo recombineering of scFv into IgG molecules. J Immunol Methods. 431:22-30.
Buldun, C. M., Jean, J., Bedford, M. R., Howarth, M., 2018, SnoopLigase catalyzes peptide-peptide locking and enables solid-phase conjugate isolation. J Am Chem Soc. 140(8), 3008-3018.
Caspi, J. et al., 2003, Distribution of split DnaE inteins in cyanobacteria. Mol Microbiol. 50:1569-1577.
Chen, W. and Georgiou, G., 2002, Cell-surface display of heterologous proteins: From high-throughput screening to environmental applications. Biotechnol Bioeng. 79:496-503.
Cho, M. S., Yee, H. & Chan, S. Establishment of a human somatic hybrid cell line for recombinant protein production. J Biomed Sci 9, 631-638(2002).
Choi, J. et al., 2006, Protein Trans-splicing and Characterization of a Split Family B-type DNA Polymerase from the Hyperthermophilic Archaeal Parasite Nanoarchaeum equitans. J Mol Biol. 356:1093-1106.
Dassa, B. et al., 2007, Trans Protein Splicing of Cyanobacterial Split Inteins in Endogenous and Exogenous Combinations. Biochemistry. 46:322-330.
Fierer, J. O., Veggiani, G., Howarth, M., 2014, SpyLigase peptide-peptide ligation polymerizes affibodies to enhance magnetic cancer cell capture. Proc Natl Acad Sci USA. 111: E1176-1181.
Gingrich, J. C., Davis, D. R., Nguyen, Q., 2000, Multiplex detection and quantitation of proteins on western blots using fluorescent probes. Biotechniques. 29(3), 636-642.
Keeble, A. H., Banerjee, A., Ferla, M. P., Reddington, S. C., Khairil Anuar, I. N. A., Howarth, M., 2017, Evolving accelerated amidation by SpyTag/SpyCatcher to analyze membrane dynamics. Ange, Chem. Int. Ed. 56:16521-16525.
Keeble, A. H., Turkki, P., Stokes, S., Khairil Anuar, I. N. A., Rahikainen, R., Hytönen, V. P., Howarth, M., 2019, Approaching infinite affinity through engineering of peptide-protein interaction. Proc Natl Acad Sci USA. 116:26526-26533.
Knappik, A., Capuano, F., Frisch, C., Ylera, F., Bonelli, F., 2009. Development of recombinant human IgA for anticardiolipin antibodies assay standardization. Annals of the New York Academy of Sciences. 1173, 190-198
Li et al., 2014, Structural analysis and optimization of the covalent association between SpyCatcher and a peptide Tag, J Mol Biol. 426(2), 309-17.
Liu, X. and Yang J., 2003, Split dnaE Genes Encoding Multiple Novel Inteins in Trichodesmium erythraeum. J Biol Chem. 278:26315-26318.
Mauro, V. P., 2018, Codon Optimization in the Production of Recombinant Biotherapeutics: Potential Risks and Considerations. BioDrugs: clinical immunotherapeutics, biopharmaceuticals and gene therapy. 32(1), 69-81.
Nguyen, G. K. T., Wang, S., Qiu, Y., Hemu, X., Lian, Y., Tam, J. P., 2014, Butelase 1 is an Asx-specific ligase enabling peptide macrocyclization and synthesis. Nat Chem Biol. 10:732-738.
Nguyen et al., 2016, Butelase-mediated cyclization and ligation of peptides and proteins. Nature Protocols. 11(10), 1977-1988.
Prassler, J., Thiel, S., Pracht, C., Polzer, A., Peters, S., Bauer, M., Norenberg, S., Stark, Y., Kolln, J., Popp, A., Urlinger, S., Enzelberger, M., 2011, HuCAL PLATINUM, a synthetic Fab library optimized for sequence diversity and superior performance in mammalian expression systems. J Mol Biol. 413(1), 261-278.
Reddington, S. C., Howarth, M., 2015, Secrets of a covalent interaction for biomaterials and biotechnology: SpyTag and SpyCatcher. Current Opinion in Chemical Biology. 29:94-99.
Schmohl, L., Schwarzer, D., 2014, Sortase-mediated ligations for the site-specific modification of proteins. Current Opinion in Chemical Biology. 22:122-128.
Shah & Muir, 2011, Split Inteins: Nature's Protein Ligases. Israel Journal of Chemistry. 51(8-9), 854-861.
Siegmund et al., 2016, Spontaneous Isopeptide Bond Formation as a Powerful Tool for Engineering Site-Specific Antibody-Drug Conjugates. Scientific Reports. 6, 39291.
Tan et al. (2016). Kinetic Controlled Tag-Catcher Interactions for Directed Covalent Protein Assembly. PLOS ONE, 11(10), e0165074.
Thiel, I. V., Volkmann, G., Pietrokovski, S., Mootz, H. D., 2014, An atypically split intein engineered for a highly efficient protein labeling. Angew Chem Int Ed Engl. 53:1306-1310.
Toplak, A., Nuljens, T., Quaedflieg, P. J.L ., Wu, B., Janssen, D. B., 2016, Peptiligase, an enzyme foe efficient chemoenzymatic peptide synthesis and cyclization in water. Adv Synth Catal. 358:32140-32147.
Veggiani, G. et al., 2016, Programmable polyproteams built using twin peptide superglues. Proc Natl Acad Sci USA 113:1202-1207.
Wu, H. et al., 1998, Protein trans-splicing by a split intein encoded in a split DnaE gene of Synechocystis sp. PCC6803. Proc Natl Acad Sci USA. 95:9226-9231.
Yumura, K. et al., 2017, Use of SpyTag/SpyCatcher to construct bispecific antibodies that target two epitopes of a single antigen. J Biochem. 162(3), 203-210.
Zakeri, B. et al., 2012, Peptide tag forming a rapid covalent bond to a protein, through engineering a bacterial adhesion. Proc Natl Acad Sci USA. 109: E690-697.
Zettler, J. et al., 2009, The Naturally Split Npu DnaE Intein Exhibits an Extraordinarily High Rate in the Protein Trans-Splicing Reaction. FEBS Letters. 553:909-914.

1. A full-length antibody comprising antigen binding fragments comprising a first binding motif at the C-terminus and an Fc fragment comprising a second binding motif at the N-terminus, wherein the first binding motif and the second binding motif are covalently conjugated to each other via protein ligation, with the proviso that if the antigen binding fragment and the Fc fragment are obtained from the same species, the Fc fragment is labeled with a detectable label.


SEQUENCE LISTING (44 sequences)

SEQ ID NO: 1 - protein, 340 aa, Streptococcus pyogenes
MKLRHLLLTGAALTSFAATTVHGETVVNGAKLTVTKNLDLVNSNALIPNTDFTFKIEPDTTVNEDGNKFKGVALNTPMTKVTYTNSDKGGSNTKTAEFDFSEVTFEKPGVYYYKVTEEKIDKVPGVSYDTTSYTVQVHVLWNEEQQKPVATYIVGYKEGSKVPIQFKNSLDSTTLTVKKKVSGTGGDRSKDFNFGLTLKANQYYKASEKVMIEKTTKGGQAPVQTEASIDQLYHFTLKDGESIKVTNLPVGVDYVVTEDDYKSEKYTTNVEVSPQDGAVKNIAGNSTEQETSTDKDMTITFTNKKDFEVPTGVAMTVAPYIALGIVAVGGALYFVKKKNA

SEQ ID NO: 2 - DNA, 1023 bp, Streptococcus pyogenes
  source 1..1023: mol_type = other DNA
atgaaattacgtcacttactattaacgggagcagccctaactagttttgctgctacaacagttcacggggagactgttgtaaacggagccaaactaacagttacaaaaaaccttgatttagttaatagcaatgcattaattccaaatacagattttacatttaaaatcgaacctgatactactgtcaacgaagacggaaataagtttaaaggtgtagctttgaacacaccgatgactaaagtcacttacaccaattcagataaaggtggatcaaatacgaaaactgcagaatttgatttttcagaagttacttttgaaaaaccaggtgtttattattacaaagtaactgaggagaagatagataaagttcctggtgtttcttatgatacaacatcttacactgttcaagttcatgtcttgtggaatgaagagcaacaaaaaccagtagctacttatattgttggttataaagaaggtagtaaggtgccaattcagttcaaaaatagcttagattctactacattaacggtgaagaaaaaagtttcaggtaccggtggagatcgctctaaagattttaattttggtctgactttaaaagcaaatcagtattataaggcgtcagaaaaagtcatgattgagaagacaactaaaggtggtcaagctcctgttcaaacagaggctagtatagatcaactctatcattttaccttgaaagatggtgaatcaatcaaagtcacaaatcttccagtaggtgtggattatgttgtcactgaagacgattacaaatcagaaaaatatacaaccaacgtggaagttagtcctcaagatggagctgtaaaaaatatcgcaggtaattcaactgaacaagagacatctactgataaagatatgaccattacttttacaaataaaaaagactttgaagtgccaacaggagtagcaatgactgtggcaccatatattgctttaggaattgtagcagttggtggagctctttactttgttaaaaagaaaaatgcttaa

SEQ ID NO: 3 - protein, 674 aa, Enterococcus faecalis
MTKSVKFLVLLLVMILPIAGALLIGPISFGAELSKSSIVDKVELDHTTLYQGEMTSIKVSFSDKENQKIKPGDTITLTLPDALVGMTENDSSPRKINLNGLGEVFIYKDHVVATFNEKVESLHNVNGHFSFGIKTLITNSSQPNVIETDFGTATATQRLTIEGVTNTETGQIERDYPFFYKVGDLAGESNQVRWFLNVNLNKSDVTEDISIADRQGSGQQLNKESFTFDIVNDKETKYISLAEFEQQGYGKIDFVTDNDFNLRFYRDKARFTSFIVRYTSTITEAGQHQATFENSYDINYQLNNQDATNEKNTSQVKNVFVEGEASGNQNVEMPTEESLDIPLETIDEWEPKTPTSEQATETSEKTDTTETAESSQPEVHVSPTEEENPDEGETLGTIEPIIPEKPSVTTEENGTTETAESSQPEVHVSPTEEENPDESETLGTIEPIIPEKPSVTTEENGTTETAESSQPEVHVSPAEEENPDESETLGTILPILPEKPSVTTEENGTTETAESSQPEVHVSPTEEENPDESETLGTIAPIIPEKPSVTTEENGITETAESSQPEVHVSPTKEITTTEKKQPSTETTVEKNKNVTSKNQPQILNAPLNTLKNEGSPQLAPQLLSEPIQKLNEANGQRELPKTGTTKTPFMLIAGILASTFAVLGVSYLQIRKN

SEQ ID NO: 4 - DNA, 2025 bp, Enterococcus faecalis
  source 1..2025: mol_type = other DNA
atgacaaaaagtgtaaaatttttagtgttactgttggtaatgattctaccaattgcgggggcgttattgattggtccaatttcgtttggcgccgaattgagcaaaagttcaatcgttgacaaagtagaattagatcacactactttatatcaaggagagatgacctcaattaaagtatcttttagtgacaaagaaaatcagaaaataaaacctggagatactattactttaactttaccagacgcactagttggaatgaccgagaacgatagttcaccacgaaaaatcaatttaaatggtttaggggaagtttttatctataaagatcatgttgtagcaacatttaacgaaaaagttgaatctttacataatgtgaatgggcatttttctttcgggattaaaacgcttatcaccaatagttctcaaccgaatgtgatagaaacggatttcggaacagcaacggcgactcaacgtttgacgattgaaggagtgactaacacagagactggccaaattgagcgagactatccgtttttttataaagtaggcgatttggctggagagtcaaatcaagtacgttggtttttaaatgtgaacctcaataaatccgatgtcacagaagatatttcaattgcggatcgacaaggaagtggtcaacaattaaataaagagagttttacatttgatattgtgaatgacaaagaaactaaatatatttcacttgccgagtttgagcaacaaggttatggcaaaattgacttcgtaacagataatgactttaacttacgtttttatcgggataaagcacgctttacttcctttatcgtccgttacacttcgacaatcacagaagcaggccaacatcaagcaacatttgaaaatagttatgacatcaattatcaactaaacaatcaagacgcaacgaatgaaaaaaatacatcacaggttaaaaatgtttttgtagaaggcgaggcaagcggcaatcaaaatgtggaaatgccaacagaagaaagtctagacattcctttagagacaatagatgaatgggaaccaaagacacctacttcggaacaggcaacagaaacaagtgaaaagacagacacaacagaaaccgcagaaagcagccaaccagaagttcatgtctcaccaacagaagaagaaaatccagatgaaggtgaaacactaggcacgattgagccaatcatacctgaaaaaccaagtgtgacaactgaagagaatggcacgacagaaactgcagaaagcagccaaccagaagttcatgtctcaccaacagaagaagaaaatccagatgaaagtgaaacactaggcacgattgagccaatcatacctgaaaaaccaagtgtgacaactgaagagaacggcacaacagaaaccgcagaaagcagccaaccagaagttcatgtctcaccagcggaagaagaaaatccagatgaaagtgaaacgttaggtacaattttaccaatcctacctgaaaaaccaagtgtgacaactgaagagaatggcacaacggaaactgcagaaagcagtcaaccagaagtccatgtgtcgccaacggaagaagaaaatccagatgaaagtgaaacactaggcacgattgcaccaatcatacctgaaaaaccaagcgtaacaactgaagagaatggtataacggaaacggcagaaagcagccagccagaagttcatgtctcaccaacaaaagaaattactacaactgagaaaaaacagccatccacagaaacaactgtggagaaaaataaaaatgttacatcaaaaaatcaaccacaaatactaaacgctccattaaatacattgaaaaatgaaggaagcccacagttggctccccaactgcttagtgaaccaattcaaaaattaaatgaagcaaacgggcaacgagaacttcccaaaacaggcacaacaaaaacaccgtttatgctaatagcaggaatactggcaagtacatttgccgttttaggtgtaagttatctacaaatcagaaagaattaa

SEQ ID NO: 5 - protein, 303 aa, Staphylococcus aureus
GSARDISSTNVTDLTVSPSKIEDGGKTTVKMTFDDKNGKIQNGDMIKVAWPTSGTVKIEGYSKTVPLTVKGEQVGQAVITPDGATITFNDKVEKLSDVSGFAEFEVQGRNLTQTNTSDDKVATITSGNKSTNVTVHKSEAGTSSVFYYKTGDMLPEDTTHVRWFLNINNEKSYVSKDITIKDQIQGGQQLDLSTLNINVTGTHSNYYSGQSAITDFEKAFPGSKITVDNTKNTIDVTIPQGYGSYNSFSINYKTKITNEQQKEFVNNSQAWYQEHGKEEVNGKSFNHTVHNINANAGIEGTVK

SEQ ID NO: 6 - protein, 102 aa, Streptococcus pyogenes
MTIEEDSATHIKFSKRDIDGKELAGATMELRDSSGKTISTWISDGQVKDFYLMPGKYTFVETAAPDGYEVATAITFTVNEQGQVTVNGKATKGDAHIVMVDA

SEQ ID NO: 7 - protein, 13 aa, synthetic construct
  REGION 1..13: note = SpyTag
AHIVMVDAYKPTK

SEQ ID NO: 8 - protein, 129 aa, synthetic construct
  REGION 1..129: note = SpyCatcher
DYDIPTTENLYFQGAMVDTLSGLSSEQGQSGDMTIEEDSATHIKFSKRDEDGKELAGATMELRDSSGKTISTWISDGQVKDFYLYPGKYTFVETAAPDGYEVATAITFTVNEQGQVTVNGKATKGDAHI

SEQ ID NO: 9 - protein, 84 aa, synthetic construct
  REGION 1..84: note = SpyCatcher (short)
GDSATHIKFSKRDEDGKELAGATMELRDSSGKTISTWISDGQVKDFYLYPGKYTFVETAAPDGYEVATAITFTVNEQGQVTVNG

SEQ ID NO: 10 - protein, 331 aa, Staphylococcus aureus
MNKNVLKFMVFIMLLNIITPLFNKNEAFAARDISSTNVTDLTVSPSKIEDGGKTTVKMTFDDKNGKIQNGDMIKVAWPTSGTVKIEGYSKTVPLTVKGEQVGQAVITPDGATITFNDKVEKLSDVSGFAEFEVQGRNLTQTNTSDDKVATITSGNKSTNVTVHKSEAGTSSVFYYKTGDMLPEDTTHVRWFLNINNEKSYVSKDITIKDQIQGGQQLDLSTLNINVTGTHSNYYSGQSAITDFEKAFPGSKITVDNTKNTIDVTIPQGYGSYNSFSINYKTKITNEQQKEFVNNSQAWYQEHGKEEVNGKSFNHTVHNINANAGIEGTVKG

SEQ ID NO: 11 - protein, 102 aa, Streptococcus pyogenes; identical to SEQ ID NO: 6

SEQ ID NO: 12 - DNA, 309 bp, Streptococcus pyogenes
  source 1..309: mol_type = other DNA
atgacaattgaagaagatagtgctacccatattaaattctcaaaacgtgatattgacggcaaagagttagctggtgcaactatggagttgcgtgattcatctggtaaaactattagtacatggatttcagatggacaagtgaaagatttctacctgatgccaggaaaatatacatttgtcgaaaccgcagcaccagacggttatgaggtagcaactgctattacctttacagttaatgagcaaggtcaggttactgtaaatggcaaagcaactaaaggtgacgctcatattgtcatggttgatgcttga

SEQ ID NO: 13 - protein, 893 aa, Streptococcus pneumoniae
MLNRETHMKKVRKIFQKAVAGLCCISQLTAFSSIVALAETPETSPAIGKVVIKETGEGGALLGDAVFELKNNTDGTTVSQRTEAQTGEAIFSNIKPGTYTLTEAQPPVGYKPSTKQWTVEVEKNGRTTVQGEQVENREEALSDQYPQTGTYPDVQTPYQIIKVDGSEKNGQHKALNPNPYERVIPEGTLSKRIYQVNNLDDNQYGIELTVSGKTVYEQKDKSVPLDVVILLDNSNSMSNIRNKNARRAERAGEATRSLIDKITSDSENRVALVTYASTIFDGTEFTVEKGVADKNGKRLNDSLFWNYDQTSFTTNTKDYSYLKLTNDKNDIVELKNKVPTEAEDHDGNRLMYQFGATFTQKALMKADEILTQQARQNSQKVIFHITDGVPTMSYPINFNHATFAPSYQNQLNAFFSKSPNKDGILLSDFITQATSGEHTIVRGDGQSYQMFTDKTVYEKGAPAAFPVKPEKYSEMKAAGYAVIGDPINGGYIWLNWRESILAYPFNSNTAKITNHGDPTRWYYNGNIAPDGYDVFTVGIGINGDPGTDEATATSFMQSISSKPENYTNVTDTTKILEQLNRYFHTIVTEKKSIENGTITDPMGELIDLQLGTDGRFDPADYTLTANDGSRLENGQAVGGPQNDGGLLKNAKVLYDTTEKRIRVTGLYLGTDEKVTLTYNVRLNDEFVSNKFYDTNGRTTLHPKEVEQNTVRDFPIPKIRDVRKYPEITISKEKKLGDIEFIKVNKNDKKPLRGAVFSLQKQHPDYPDIYGAIDQNGTYQNVRTGEDGKLTFKNLSDGKYRLFENSEPAGYKPVQNKPIVAFQIVNGEVRDVTSIVPQDIPAGYEFTNDKHYITNEPIPPKREYPRTGGIGMLPFYLIGCMMMGGVLLYTRKHP

SEQ ID NO: 14 - DNA, 2682 bp, Streptococcus pneumoniae
  source 1..2682: mol_type = other DNA
atgctgaaccgcgaaacccatatgaaaaaagtaagaaagatatttcagaaggcagttgcaggactgtgctgtatatctcagttgacagctttttcttcgatagttgctttagcagaaacgcctgaaaccagtccagcgataggaaaagtagtgattaaggagacaggcgaaggaggagcgcttctaggagatgccgtctttgagttgaaaaacaatacggatggcacaactgtttcgcaaaggacagaggcgcaaacaggagaagcgatattttcaaacataaaacctgggacatacaccttgacagaagcccaacctccagttggttataaaccctctactaaacaatggactgttgaagttgagaagaatggtcggacgactgtccaaggtgaacaggtagaaaatcgagaagaggctctatctgaccagtatccacaaacagggacttatccagatgttcaaacaccttatcagattattaaggtagatggttcggaaaaaaacggacagcacaaggcgttgaatccgaatccatatgaacgtgtgattccagaaggtacactttcaaagagaatttatcaagtgaataatttggatgataaccaatatggaatcgaattgacggttagtgggaaaacagtgtatgaacaaaaagataagtctgtgccgctggatgtcgttatcttgctcgataactcaaatagtatgagtaacattcgaaacaagaatgctcgacgtgcggaaagagctggtgaggcgacacgttctcttattgataaaattacatctgattcagaaaatagggtagcgcttgtgacttatgcttccactatctttgatgggaccgagtttacagtagaaaaaggggtagcagataaaaacggaaagcgattgaatgattctcttttttggaattatgatcagacgagttttacaaccaataccaaagattatagttatttaaagctgactaatgataagaatgacattgtagaattaaaaaataaggtacctaccgaggcagaagaccatgatggaaatagattgatgtaccaattcggtgccacttttactcagaaagctttgatgaaggcagatgagattttgacacaacaagcgagacaaaatagtcaaaaagtcattttccatattacggatggtgtcccaactatgtcgtatccgattaattttaatcatgctacgtttgctccatcatatcaaaatcaactaaatgcattttttagtaaatctcctaataaagatggaatactattaagtgattttattacgcaagcaactagtggagaacatacaattgtacgcggagatgggcaaagttaccagatgtttacagataagacagtttatgaaaaaggtgctcctgcagctttcccagttaaacctgaaaaatattctgaaatgaaggcggctggttatgcagttataggcgatccaattaatggtggatatatttggcttaattggagagagagtattctggcttatccgtttaattctaatactgctaaaattaccaatcatggtgaccctacaagatggtactataacgggaatattgctcctgatgggtatgatgtctttacggtaggtattggtattaacggagatcctggtacggatgaagcaacggctactagttttatgcaaagtatttctagtaaacctgaaaactataccaatgttactgacacgacaaaaatattggaacagttgaatcgttatttccacaccatcgtaactgaaaagaaatcaattgagaatggtacgattacagatccgatgggtgagttaattgatttgcaattgggcacagatggaagatttgatccagcagattacactttaactgcaaacgatggtagtcgcttggagaatggacaagctgtaggtggtccacaaaatgatggtggtttgttaaaaaatgcaaaagtgctctatgatacgactgagaaaaggattcgtgtaacaggtctgtaccttggaacggatgaaaaagttacgttgacctacaatgttcgtttgaatgatgagtttgtaagcaataaattttatgataccaatggtcgaacaaccttacatcctaaggaagtagaacagaacacagtgcgcgacttcccgattcctaagattcgtgatgtgcggaagtatccagaaatcacaatttcaaaagagaaaaaacttggtgacattgagtttattaaggtcaataaaaatgataaaaaaccactgagaggtgcggtctttagtcttcaaaaacaacatccggattatccagatatttatggagctattgatcaaaatggcacttatcaaaatgtgagaacaggtgaagatggtaagttgacctttaaaaatctgtcagatgggaaatatcgattatttgaaaattctgaaccagctggttataaacccgttcaaaataagcctatcgttgccttccaaatagtaaatggagaagtcagagatgtgacttcaatcgttccacaagatataccagcgggttacgagtttacgaatgataagcactatattaccaatgaacctattcctccaaagagagaatatcctcgaactggtggtatcggaatgttgccattctatctgataggttgcatgatgatgggaggagttctattatacacacggaaacatccgtaa

SEQ ID NO: 15 - protein, 738 aa, Streptococcus intermedius
MKKRRGQFFKSAISFLVVFLMVMVSIIYPSSKIKADGFPNDATGVSPNGKYYSAGRENRLGMVTSDELHTATELFGFCMANSKKYPGYDSKKDEYFGVYEQILNLNKESFNKLVRDNHTYGNIPTSPEELWDKVSKLIYIYLKDPTNVIGQAGWTNPQDAMYEFYTVVQQEIWRYTDGQKVDKDTNSYLYYKYSKQGQKAVYLLRDAVNSISIPSNFELRGYKPEWVQGQKGYQAIVTGRLKVDQPVGEIKTTVTAGGKTSSENDIATLKAQDVIGGVEVSDKITYSGLYPNTEYDVIGEIYEVKDGELVNPGRPVSVVNSGDDLKTDATGKGKWTLNFGKLDLEAGKSYVVFEKVVSLKNVIDTDGDGKPDKKQELSHNDPKDKSQTFTILPKEIVEQDVVFSKVNVAGEEIAGAKIQLKDAQGQVVHSWTSKAGQSETVKLKAGTYTFHEASAPTGYLAVTDITFEVDVQGKVTVKDANGNGVKADGNKLTVTDQAAPSVPNEQDVVFSKVNVAGEEIAGAKIQLKDAQGQVVHSWTSKAGQSETVKLKAGTYTFHEASAPTGYLAVTDITFEVDVQGKVTVKDANGNGVKADGNKLTVTDQAAPSVPNEQDVVFSKVNVAGEEIAGAKIQLKDAQGQVVHSWTSKAGQSETVKLKAGTYTFHEASAPTGYLAVTDITFEVDVQGKVTVKDANGNGVKADGNKLTVTDQAAPSVPNEQDVVFSKVNVAGEEIAGAK

SEQ ID NO: 16 - DNA, 2215 bp, Streptococcus intermedius
  source 1..2215: mol_type = other DNA
atgaaaaagagaagaggacaatttttcaaaagtgcaatttcgtttttggttgtatttttgatggtaatggtaagtatcatttacccatcttcaaaaattaaagcagatggatttcctaatgatgctacgggagtatcgccaaatggtaaatattactcggcagggagagaaaaccgtttaggaatggttacatcagatgaattgcatacagctacagaattattcggtttttgtatggcaaatagcaagaaatatccaggatatgattcaaaaaaggatgagtattttggggtgtatgaacaaatcttaaaccttaataaagaaagctttaataagcttgttagagataatcatacgtatggtaacattcctacaagtccagaggaactttgggataaagtatctaaactgatttatatttatttgaaagaccctacaaatgttattggacaagctgggtggacgaatccacaggatgcaatgtatgaattttatactgttgtacaacaggaaatatggcgttatacagatggacaaaaggtggataaagacaccaattcatatttgtattataaatattcaaaacaaggtcaaaaagcagtgtacttactgcgtgacgctgtgaatagcatcagtatacctagtaattttgaacttcgtggctataaacctgaatgggttcaaggtcaaaaaggataccaagctattgtaactggtagattgaaagtagatcaacctgtcggggaaataaagactacagtaacagcaggtggaaaaacctcaagtgaaaacgacattgctacattgaaggcgcaagacgttataggtggggttgaagtctctgataagataacatatagtggtctttatccaaatacagaatatgatgttataggtgaaatttacgaagtaaaagatggagaacttgttaatccaggacgaccggtttctgtagtcaatagtggtgacgatttaaaaacagatgcaacaggaaaagggaaatggacattaaactttggaaagcttgatttagaagcaggaaaatcctatgtggtctttgaaaaagttgtttcattaaaaaacgtgatagatacagatggagatggaaaaccggataaaaaacaagaactatcgcataatgatccaaaagataaatcgcaaacatttacaattttacctaaggaaatagttgaacaagacgttgtcttcagtaaggtgaatgtggctggtgaagaaatcgctggtgcgaagatccaactgaaggatgcgcaaggtcaagttgttcattcctggacttctaaagcgggtcaaagtgaaacggtcaaattgaaagctggcacctatactttccatgaagcatccgctccgactggttacttggccgtaacggatatcacattcgaagtagatgttcaaggaaaagtgacggttaaggatgccaacggcaatggtgttaaggcggatggtaataagttaacggtgaccgatcaagctgctcctagcgtaccgaatgaacaagacgttgtcttcagtaaggtgaatgtggctggtgaagaaatcgctggtgcgaagatccaactgaaggatgcgcaaggtcaagttgttcattcctggacttctaaagcgggtcaaagtgaaacggtcaaattgaaagctggcacctatactttccatgaagcatccgctccgactggttacttggccgtaacggatatcacattcgaagtagatgttcaaggaaaagtgacggttaaggatgccaacggcaatggtgttaaggcggatggtaataagttaacggtgaccgatcaagctgctcctagcgtaccgaatgaacaagacgttgtcttcagtaaggtgaatgtggctggtgaagaaatcgctggtgcgaagatccaactgaaggatgcgcaaggtcaagttgttcattcctggacttctaaagcgggtcaaagtgaaacggtcaaattgaaagctggcacctatactttccatgaagcatccgctccgactggttacttggccgtaacggatatcacattcgaagtagatgttcaaggaaaagtgacggttaaggatgccaacggcaatggtgttaaggcggatggtaataagttaacggtgaccgatcaagctgctcctagcgtaccgaatgaacaagacgttgtcttcagtaaggtgaatgtggctggtgaagaaatcgctggtgcgaaga

SEQ ID NO: 17 - protein, 5 aa, synthetic construct
  REGION 1..5: note = synthetic
  VARIANT 3: note = X is any amino acid
LPXTG

SEQ ID NO: 18 - protein, 6 aa, synthetic construct
  REGION 1..6: note = synthetic
LPTGAA

SEQ ID NO: 19 - protein, 6 aa, synthetic construct
  REGION 1..6: note = Synthetic
LPTGGG

SEQ ID NO: 20 - protein, 6 aa, synthetic construct
  REGION 1..6: note = synthetic
LPKTGG

SEQ ID NO: 21 - protein, 5 aa, synthetic construct
  REGION 1..5: note = synthetic
LPETG

SEQ ID NO: 22 - protein, 5 aa, synthetic construct
  REGION 1..5: note = synthetic
  VARIANT 3: note = X is any amino acid
LPXTG

SEQ ID NO: 23 - protein, 6 aa, synthetic construct
  REGION 1..6: note = synthetic
  VARIANT 3: note = X is any amino acid
  VARIANT 6: note = X is any amino acid
LPXTGX

SEQ ID NO: 24 - skipped

SEQ ID NO: 25 - protein, 40 aa, synthetic construct
  REGION 1..40: note = synthetic
MGSSHHHHHHSSGLVPRGSVPTIVMVDAYKRYKGSGESGK

SEQ ID NO: 26 - protein, 139 aa, synthetic construct
  REGION 1..139: note = synthetic
MSYYHHHHHHDYDIPTTENLYFQGAMVTTLSGLSGEQGPSGDMTTEEDSATHIKFSKRDEDGRELAGATMELRDSSGKTISTWISDGHVKDFYLYPGKYTFVETAAPDGYEVATAITFTVNEQGQVTVNGEATKGDAHT

SEQ ID NO: 27 - protein, 15 aa, synthetic construct
  REGION 1..15: note = synthetic
VPTIVMVDAYKRYKS

SEQ ID NO: 28 - protein, 120 aa, synthetic construct
  REGION 1..120: note = SpyCatcher002
AMVTTLSGLSGEQGPSGDMTTEEDSATHIKFSKRDEDGRELAGATMELRDSSGKTISTWISDGHVKDFYLYPGKYTFVETAAPDGYEVATAITFTVNEQGQVTVNGEATKGDAHTGSSGS

SEQ ID NO: 29 - protein, 13 aa, synthetic construct
  REGION 1..13: note = synthetic
AHIVMVDAYKPTK

SEQ ID NO: 30 - protein, 10 aa, synthetic construct
  REGION 1..10: note = synthetic
AHIVMVDAYK

SEQ ID NO: 31 - protein, 15 aa, synthetic construct
  REGION 1..15: note = synthetic
EPKSCDKTHTCPPCP

SEQ ID NO: 32 - protein, 5 aa, synthetic construct
  REGION 1..5: note = synthetic
GGGGS

SEQ ID NO: 33 - protein, 10 aa, synthetic construct
  REGION 1..10: note = synthetic
ATHIKFSKRD

SEQ ID NO: 34 - protein, 14 aa, synthetic construct
  REGION 1..14: note = SpyTag002
VPTIVMVDAYKRYK

SEQ ID NO: 35 - protein, 12 aa, synthetic construct
  REGION 1..12: note = SnoopTag
KLGDIEFIKVNK

SEQ ID NO: 36 - protein, 112 aa, synthetic construct
  REGION 1..112: note = SnoopCatcher
KPLRGAVFSLQKQHPDYPDIYGAIDQNGTYQNVRTGEDGKLTFKNLSDGKYRLFENSEPAGYKPVQNKPIVAFQIVNGEVRDVTSIVPQDIPATYEFTNGKHYITNEPIPPK

SEQ ID NO: 37 - protein, 12 aa, synthetic construct
  REGION 1..12: note = SnoopTagJr
KLGSIEFIKVNK

SEQ ID NO: 38 - protein, 23 aa, synthetic construct
  REGION 1..23: note = DogTag
DIPATYEFTNGKHYITNEPIPPK

SEQ ID NO: 39 - protein, 16 aa, synthetic construct
  REGION 1..16: note = Isopeptag
TDKDMTITFTNKKDAE

SEQ ID NO: 40 - protein, 282 aa, synthetic construct
  REGION 1..282: note = Split Spy0128
ATTVHGETVVNGAKLTVTKNLDLVNSNALIPNTDFTFKIEPDTTVNEDGNKFKGVALNTPMTKVTYTNSDKGGSNTKTAEFDFSEVTFEKPGVYYYKVTEEKIDKVPGVSYDTTSYTVQVHVLWNEEQQKPVATYIVGYKEGSKVPIQFKNSLDSTTLTVKKKVSGTGGDRSKDFNFGLTLKANQYYKASEKVMIEKTTKGGQAPVQTEASIDQLYHFTLKDGESIKVTNLPVGVDYVVTEDDYKSEKYTTNVEVSPQDGAVKNIAGNSTEQETSTDKDMTI

SEQ ID NO: 41 - protein, 13 aa, synthetic construct
  REGION 1..13: note = SdyTag
DPIVMIDNDKPIT

SEQ ID NO: 42 - protein, 134 aa, synthetic construct
  REGION 1..134: note = SdyCatcherDANG short
MGSSHHHHHHSSGLVPRGSHMASMTGGQQMGRGSSGLSGETGQSGNTTIEEDSTTHVKFSKRDANGKELAGAMIELRNLSGQTIQSWISDGTVKVFYLMPGTYQFVETAAPEGYELAAPITFTIDEKGQIWVDS

SEQ ID NO: 43 - protein, 16 aa, synthetic construct
  REGION 1..16: note = synthetic
RGVPHIVMVDAYKRYK

SEQ ID NO: 44 - protein, 113 aa, synthetic construct
  REGION 1..113: note = synthetic
VTTLSGLSGEQGPSGDMTTEEDSATHIKFSKRDEDGRELAGATMELRDSSGKTISTWISDGHVKDFYLYPGKYTFVETAAPDGYEVATPIEFTVNEDGQVTVDGEATEGDAHT